(19) United States
(12) Patent Application Publication
(10) Pub. No.: US 2025/0157590 A1
(43) Pub. Date: May 15, 2025

(54) SYSTEMS AND METHODS FOR CHEMICAL TOXICITY PREDICTION

(71) Applicants: NATIONAL TAIWAN UNIVERSITY, TAIPEI (TW); NATIONAL ENVIRONMENTAL RESEARCH ACADEMY, TAOYUAN CITY (TW)

(72) Inventor: YUFENG JANE TSENG, Taipei (TW)

(21) Appl. No.: 18/923,647

(22) Filed: Oct. 22, 2024

Related U.S. Application Data
(60) Provisional application No. 63/598,148, filed on Nov. 13, 2023.

(51) Int. Cl. G16C 20/30

(52) U.S. Cl. CPC G16C 20/30

(57) ABSTRACT

The present disclosure relates to systems and methods for chemical toxicity prediction. The methods of the present disclosure comprise: receiving test data from an analysis instrument; selecting a candidate chemical according to the test data; determining a hazard translated level and a hazard evaluation level of the candidate chemical according to a molecular fingerprint of the candidate chemical; and predicting the toxicity of the candidate chemical by using a quantitative structure-activity relationship (QSAR) model based on the hazard translated level and the hazard evaluation level.

### CROSS-REFERENCE TO RELATED APPLICATIONS

The present application claims the priority from the U.S. provisional patent application Ser. No. 63/598,148 filed Nov. 13, 2023, and the disclosure of which is incorporated herein by reference in its entirety.

### BACKGROUND OF THE INVENTION

### 1. Field of the Invention

The present disclosure relates to systems and methods for chemical toxicity prediction; more specifically, the present disclosure relates to systems and methods for toxicity prediction of chemicals in industrial wastes, wastewater, discharged water, exhaust gases, products or byproducts.

### 2. Description of the Related Art

In recent years, the United Nations has promoted sustainable development goals (SDGs), and China is promoting the 2050 net-zero emission pathway policy and transformation policy. Under the green consumption and net-zero policies, many industries are undergoing a transformation to gradually develop and apply emerging green consumer chemicals that are more energy-saving and more efficient. Since these emerging green consumer chemicals are applied to various industrial processes as well as researches and developments that are innovate and keep up with the times, it is necessary for toxicity hazard evaluation of such emerging chemicals. However, there is a lack of toxicity testing and relevant hazard data for emerging green consumer chemicals. Therefore, applicable data are collected by using internationally established chemical toxicity databases, and the structure and toxicity of a chemical can be simulated and predicted through data mining technology and artificial intelligence technology. This will save research funds, a lot of time and experimental animals and will benefit the industrial transformation, the competitiveness improvement, and the business opportunities of domestic green consumer chemical net-zero emission transformation.

### SUMMARY OF THE INVENTION

In the present disclosure, operation systems and methods for green consumer chemical toxicity prediction are established through the collection and integration of domestic and foreign databases combined with the establishment of a quantitative structure-activity relationship (QSAR) model cascaded with analysis instruments, so as to achieve the promotion of green chemistry.

Some embodiments of the present disclosure provide a method for chemical toxicity prediction. The method comprises: receiving test data from an analysis instrument; selecting a candidate chemical according to the test data; determining a hazard translated level and a hazard evaluation level of the candidate chemical according to a molecular fingerprint of the candidate chemical; and predicting the toxicity of the candidate chemical by using a quantitative structure-activity relationship (QSAR) model based on the hazard translated level and the hazard evaluation level.

Some embodiments of the present disclosure provide a system for chemical toxicity prediction. The system comprises: a database; a host comprising a memory and a processor; and an analysis instrument. The memory stores instructs so that the host executes the following operations: receiving test data of a sample from the analysis instrument; selecting a candidate chemical from the database according to the test data; determining a hazard translated level and a hazard evaluation level of the candidate chemical from the database according to a molecular fingerprint of the candidate chemical; and predicting the toxicity of the candidate chemical by using a quantitative structure-activity relationship (QSAR) model based on the hazard translated level and the hazard evaluation level.

### BRIEF DESCRIPTION OF THE DRAWINGS

According to detailed descriptions of the following reference drawings, the present disclosure will become more understandable. It is noted that various features may not be plotted in proportions. In actual, for the clear description, the sizes of various features can be arbitrarily increased or decreased.

FIG. 1 is a schematic diagram showing a workflow according to one embodiment of the present disclosure.

FIG. 2 is a schematic diagram showing systematic challenges of sustainable production and consumption in green consumerism.

FIG. 3 is a schematic diagram showing 12 principles and introduction of green chemistry.

FIG. 4 is a schematic diagram showing a relationship between alternative testing methods in the EU REACH regulation.

FIG. 5 is a schematic diagram of an OECD QSAR Toolbox operation interface according to one embodiment of the present disclosure.

FIG. 6 is a schematic diagram showing calculation operation steps of OECD QSAR Toolbox according to one embodiment of the present disclosure.

FIG. 7 is a schematic diagram of high throughput screening of toxicity test database ToxCast.

FIG. 8 is a schematic diagram of a GreenScreen list translator flow according to one embodiment of the present disclosure.

FIG. 9 is a schematic diagram of GreenScreen benchmark level.

FIG. 10 is an illustration showing symbols of chemical globally harmonized system (GHS).

FIG. 11 is a schematic diagram showing a relationship among three sub-databases in PubChem database.

FIG. 12 is a schematic diagram of drug molecular information in ChEMBL database.

FIG. 13 is a screening flowchart of a global fluoroalkyl polyolefin database updating list.

FIG. 14 shows that 5120 toxicological experiment data from PFAS in CompTox database are stored in the JSON format according to one embodiment of the present disclosure.

FIG. 15 is a flowchart showing establishment of a SIMILES and molecular fingerprint database of per/polyfluoroalkyl substances according to one embodiment of the present disclosure.

FIG. 16 is a schematic diagram showing work database model association according to one embodiment of the present disclosure.

FIG. 17 is a data presentation schematic diagram of chemical information and hazard data retrieved from a work database according to one embodiment of the present disclosure.

FIG. 18 is a schematic diagram of data presentation interface design according to one embodiment of the present disclosure.

FIG. 19 is a schematic diagram of data presentation interface design according to one embodiment of the present disclosure.

FIG. 20 is a flowchart showing an algorithm according to one embodiment of the present disclosure.

FIG. 21 is a flowchart showing the preprocessing of a training dataset according to one embodiment of the present disclosure.

FIG. 22 is a schematic diagram showing conversion of raw data according to one embodiment of the present disclosure.

FIG. 23 is a total ion chromatogram according to one embodiment of the present disclosure.

FIG. 24-FIG. 27 show optimized parameter test results under different conditions according to certain embodiments of the present disclosure.

FIG. 28 shows a computer system according to one embodiment of the present disclosure.

### DETAILED DESCRIPTION

The following disclosed contents are used for implementing many different embodiments or examples of different features of the provided subject. Next, specific examples for operations, components and configurations will be described to simplify the present disclosure. Of course, these operations, components, and configurations are only examples and not intended to limit the present disclosure. For example, a first operation executed before or after a second operation in the description may include embodiments in which the first and second operations are executed together, and may also include embodiments in which additional operations are executed between the first and second operations. For example, in the following description, the formation of the first feature above, on, or within the second feature may include embodiments formed by direct contact between the first feature and the second feature, and may also include embodiments where additional features can be formed between the first feature and the second feature so that the first feature and the second feature do not come into direct contact. In addition, the present disclosure can repeatedly refer to reference numbers and/or letters. This repetition is for the purpose of simplicity and clarity, and does not specify the relationships between the various embodiments and/or configurations discussed.

For the convenience of description, spatial relative terms such as “before”, “in the front”, “at the back”, “after” and other similar terms can be used herein to describe a relationship between one operation or feature depicted in the figures and another (multiple) element(s) or feature. The term relative time is intended to encompass different sequences of operations depicted in various figures. In addition, for the convenience of description, spatial relative terms such as “below”, “under”, “lower part”, “above”, “upper part” and other similar terms can be used herein to describe a relationship between one component or feature as depicted in the figures and another (multiple) component(s) or feature. In addition to the orientations depicted in the figures, spatial relative terms are also intended to encompass different orientations of devices in use or operation. The device can be oriented in other ways (rotating 90 degrees or in other orientations), and the spatial relative descriptor used herein can also be interpreted accordingly. For ease of description, relative terms about connection can be used herein, such as “connect”, “connected”, “connection”, “couple”, “coupled”, “communicate” and other similar terms, to describe one of operational connection, coupling or link of two components or features. The relative term used for connection is intended to cover different connections, couplings or link of devices or components. The devices or components can be directly or indirectly connected, coupled or linked to each other via another component. The devices or components can be wiredly or wirelessly connected, coupled or linked to each other.

As used herein, unless the context clearly indicates otherwise, singular terms “a/an” and “the” may include plural references. For example, unless the context clearly indicates otherwise, reference to devices may include plural devices. The terms “comprise” and “include” may indicate the presence of features, integers, steps, operations, elements and/or components, but the presence of one of the features, integers, steps, operations, elements and/or components or a combination thereof cannot be excluded. The term “and/or” may include one or more any listed items or all combinations thereof.

In addition, quantities, ratios, and other numerical values are sometimes presented in range format. In should be noted that such the range format is used for convenience and conciseness, and should be flexibly understood as including not only numerical values explicitly specified as range limits, but also all individual numerical values or sub ranges within that range, as if each numerical value and sub-range were explicitly specified.

The natures and purposes of embodiments are described in detail as follows. However, it is understood that the present disclosure provide many applicable inventive concepts, which can exhibit a wide range of multiple specific situations. Specific embodiments described only illustrate and utilize specific methods of the present invention, without limiting their scope.

In the present disclosure, high-tech industrial wastewater and discharged water may be used as test and analysis samples. The purposes of the present disclosure may be to understand analytical principles, interpret analytical data, and understand system operations. The samples disclosed in the present disclosure may also contain unknown chemical substances but no their fragment information; the present disclosure can first focus on identifiable chemicals. The present disclosure can confirm the possibility of analysis. The present disclosure can comprise analysis requirements on unknown chemical substances.

The present disclosure can integrate multiple national attention or control lists as well as multiple international organization attention or control lists. The present disclosure can combine multiple per/polyfluoroalkyl polyolefin databases and lists. By chemical structure comparison, the present disclosure can integrate chemical substance information to be stored in relevant work databases. The present disclosure can comprise relevant application programming interface (API), so as to quickly obtain characteristic information of specific chemical substances. In the present disclosure, a molecule fingerprint group compatible to PubChem can be established based on simplified molecular input line entry specification (SMILES) of chemical structures, and these information is stored in relevant work databases, and a chemical characteristic interface that is easy to read is also designed. PubChem refers to an open chemistry database of the National Institutes of Health (NIH) in the United States. The present disclosure can clear up a training dataset with 3,810 pieces of information in total based on of carcinogenic hazard endpoints. In the present disclosure, a QSAR model can be designed based on the principle provided by the Organisation for Economic Co-operation and Development (OECD), and training data processing, hazard endpoints, and predictable applicability domains of algorithms and definition are planned. In the present disclosure, a toxicity prediction model prototype can be established after searching expert opinions via an expert meeting, and a chemical characteristic input work database is predicted. For an instrument measurement and identification flow, the raw data of the instrument is converted into a public format in the present disclosure. The present disclosure comprises an instrument signal analysis flow and parameter adjustment. In the present disclosure, actual detection can be performed by test data and chemicals are identified. In the present disclosure, chemical toxicity data can be predicted by QSAR model and added into the work database.

In recent years, the United Nations has promoted SDGs, and China is promoting the 2050 net-zero emission pathway policy and transformation policy. Under the green consumption and net-zero policy, many industries are undergoing transformation, that is to say, emerging green consumer chemicals that are more energy-saving and more efficient are gradually developed and applied. Since these emerging green consumer chemicals are applied to various industrial processes as well as researches and developments that are innovate and keep up with the times, it is necessary for toxicity hazard evaluation of such emerging chemicals. However, there is a lack of toxicity testing and relevant hazard data for emerging green consumer chemicals, and therefore applicable data are collected by using an internationally established chemical toxicity database, and data mining technology and artificial intelligence technology are used to simulate structures and predict chemical toxicity, which saves research funds, a lot of time and experimental animals, is beneficial for industrial transformation and improving competitiveness, and benefits for the business opportunities of domestic green consumer chemical net-zero emission transformation.

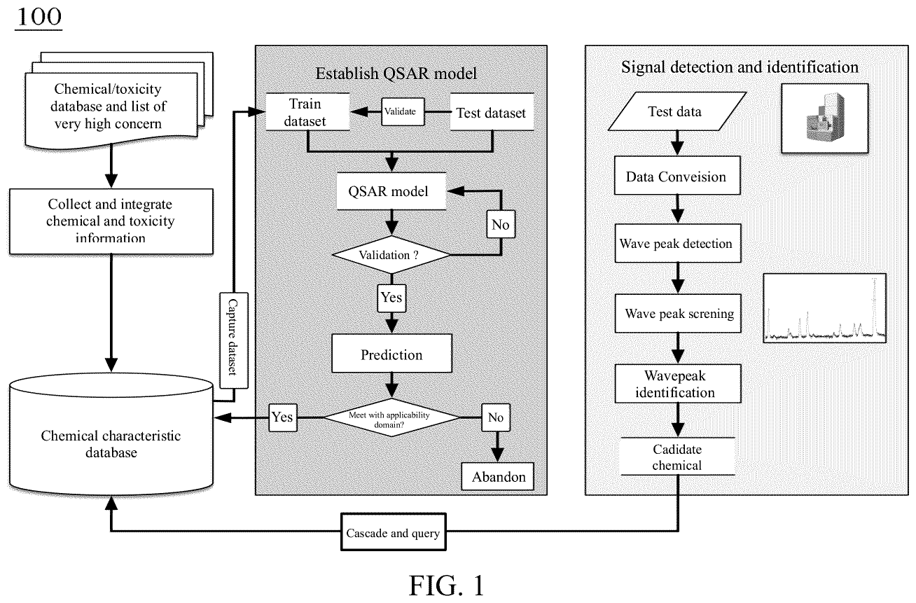

In the present disclosure, operation flows, systems, and methods for green consumer chemical toxicity prediction are established through the collection and integration of domestic and foreign databases combined with the establishment of a quantitative structure-activity relationship (QSAR) model cascaded with an analysis instrument, so as to achieve the promotion of green chemistry. The work flow 100 of the present disclosure is seen in FIG. 1. The planning work contents of a green consumer chemical toxicity prediction technology flow are described below.

First, in the present disclosure, chemical information in various databases is integrated by collecting international chemical databases and comparing by chemical structures, so as to obtain structure and characteristic information of green consumer chemicals.

Second, in the present disclosure, a green consumer chemical prediction model is established and further integrated into a chemical characteristic prediction system. A chemical molecule fingerprint group is established according to chemical structures so as to establish a chemical characteristic prediction flow, which comprises:
(1) data preprocessing;
(2) dividing the data into a chemical characteristic prediction training dataset (at least 3,000) and a validation dataset;
(3) predicting chemical characteristics after application domain evaluation.

Third, a chemical characteristic structure prediction model is established.

Fourth, an instrument measurement and an identification flow are established cascaded with a chemical characteristic prediction system and method.

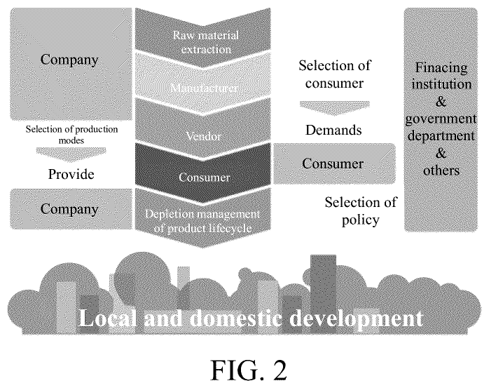

With the evolution of the times, an attitude and movement of modern society is outlined accompanied with elements of economy, society and culture, which is called green consumerism. The green consumerism is a comprehensive and responsible management process that can meet, identify, achieve and anticipate the needs of stakeholders in the aspects of maintaining environmental and natural well-being and no harm to human health. The green consumerism establishes a balance between the behavior of buyers and the profit goals of organizations, as shown in FIG. 2. Green chemistry is a discipline related to chemical products and production flows for the purpose of reducing or eliminating the production and use of harmful substances. The green chemistry exists in various stages of a chemical product life cycle, including design, manufacturing, use and disposal. In the production process of chemical products, it easily deviates from the concept of green consumerism if no supervision to cause irreversible environmental catastrophe. Thus, the concept of green chemistry must be given strong attentions in order to facilitate the entire sustainable business development.

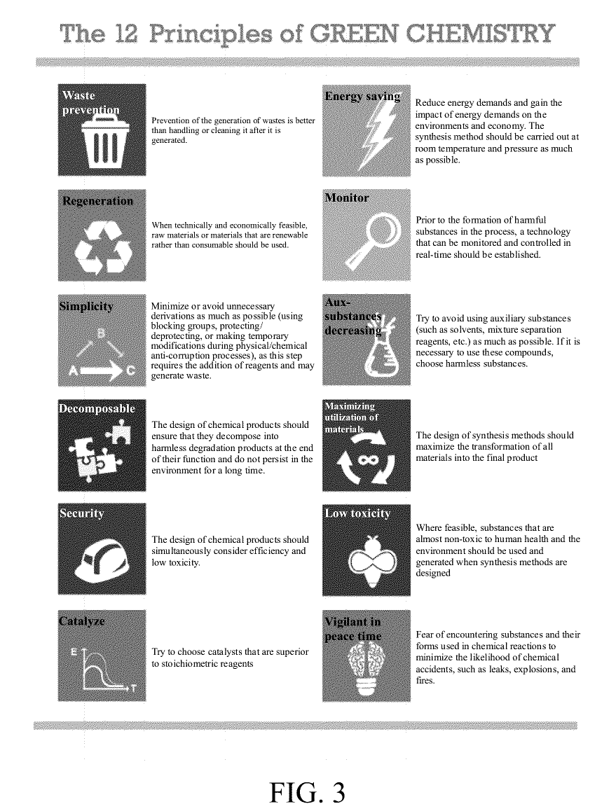

The green chemistry movement began in the early 1990s by the US Environmental Protection Agency (US EPA), aimed at encouraging industry and academia to use chemistry to prevent pollution. More specifically, the mission of green chemistry is to “promote the reduction or elimination of hazard substances in design, manufacturing and use of chemical products, or create innovative chemical technologies.” The green chemistry movement expands from the United States to Europe, Australia and Asia. From these principles, it is evident that green chemistry encompasses the concept of sustainability, rather than just pollution prevention. Green chemistry includes 12 principles that show the concept of green chemistry, as shown in FIG. 3 and Table 1. FIG. 3 can further refer to “The Inaugural Issue of The Annual Report on The Green Chemical Industry Applications and Promotion; Table 1 can further refer to “the Guidelines for the Safety Substitution of Chemical Substances in China Established by the Toxic and Chemical Substances Bureau 107 of the Environmental Protection Agency of the Executive Yuan”.

TABLE 1
China establishes a chemical safety alternative guidance
plan to classify the 12 principles of green chemistry.

| | Production process | Chemicals |
|---|---|---|
| Raw materials (reactants) | (reaction process) | (products) |
| Reduce the characteristics | (1) Reduce (no) wastes | (1) Maximize raw |
| of used raw materials: | (2) Reduce (no) | material components |
| (1)Flammability/ | derivatives | (2) No harm |
| combustibility | (3) Reduce the use of | (3) Recyclable |
| (2) Toxicity | solvents | (4) Naturally |
| (3) Effumability | (4) Time saving/ | decomposable |
| (4) Corrosivity | energy conservation | (5) Safety |
| (5) Explosibility | (available enzyme) | |
| | (5) Real-time | |
| | monitoring mechanism | |
| | (6) Safe process | |

With the advancement of risk evaluation and safety analysis of chemical substances in chemical products, the demand for rapid screening and prudent quality control continues to grow, and there is an urgent need to develop new strategies for predicting chemical characteristics. The Quantitative Structure Activity Relationship (QSAR) model is a tool convenient to use, with the principle that the interaction between small organic molecules and biomacromolecules is studied using a simulation and calculation means by virtue of physical, chemical and structural property parameters of molecules. This method can be applied to drugs, pesticides or chemicals, etc. The QSAR model is an important tool for chemical risk evaluation in cross model prediction and chemical reading.

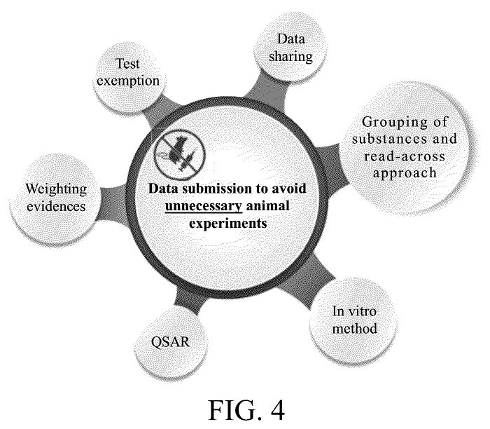

The OECD QSAR Toolbox is software jointly developed by the OECD and the European Chemical Agency (ECHA). The software can be used by government agencies, chemical industry and academic fields. The software version adopted in the present disclosure is 4.5 SP1, which is released in March 2022. The built-in database in the software includes 59 sub-databases, over 100,000 chemicals, 3 million experimental measurement data, and 902 QSAR models in four main fields, as shown in Table 2 below. Various novel chemicals have emerged in various applications, and the EU REACH regulation requires that chemical registration information must include toxicological information, however, the existing toxicological data is insufficient, and animal protection groups are calling for reducing animal experiments, and therefore, it is desired to use an alternative method in which computer calculation is utilized for animal experiments. FIG. 4 shows a relationship between test methods in the EU REACH (Registration, Evaluation, Authorization and restriction of Chemicals) regulation, which can further refer to the explanation of the Chemical Substances Login Platform 2.0 of the Chemical Substances Administration of the Ministry of Environment. At present, the REACH regulation can provide toxicological data through the alternative test method, including:
1. Use of existing data;
2. Weight of evidence;
3. QSAR;
4. In vitro methods; and
5. Grouping of substances and read-across approach.

TABLE 2
Data statistics contained in four main classifications
of ECD QSAR Toolbox 4.5 built-in database

| | | The number | The number |
|---|---|---|---|
| | The number | of experimental | of QSAR |
| Classification | of chemicals | measurement data | models |
| Physical-chemical | 50,642 | 239,949 | 28 |
| properties | | | |
| Environmental fate and | 15,356 | 171,861 | 41 |
| transmission | | | |
| Environmental | 23,137 | 1,349,467 | 688 |
| toxicology information | | | |
| Human health hazard | 45,904 | 1,263,995 | 145 |

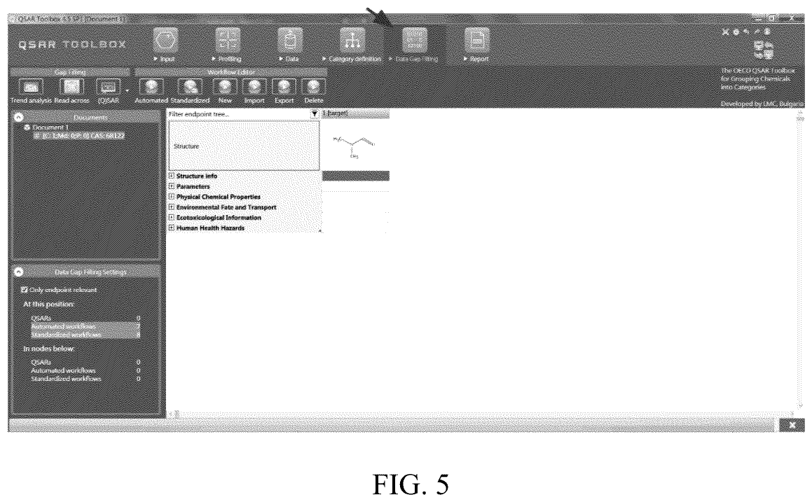

The OECD QSAR Toolbox can search the built-in database based on the CAS number or names of chemicals. If the built-in database already has toxicological data that has been tested and can be publicly obtained, it does not need to be predicted. The OECD QSAR Toolbox operation interface can refer to FIG. 5, which uses dimethylformamide (DMF; CAS number: 68-12-2) as an example.

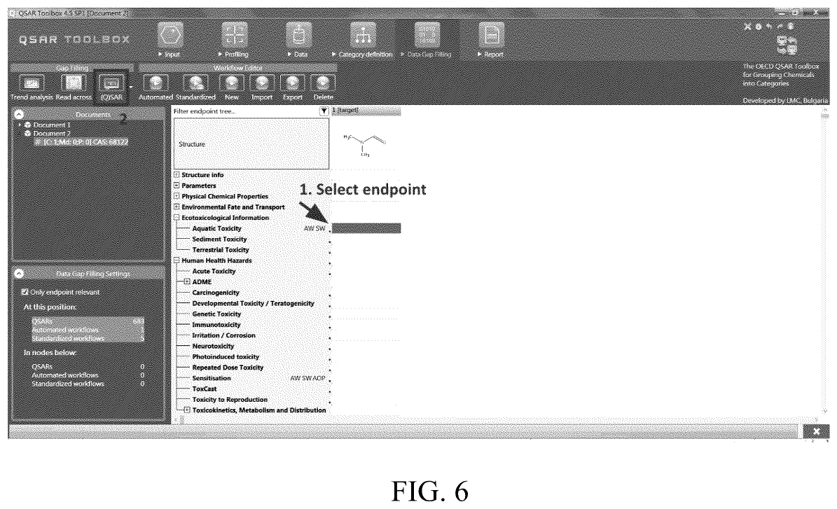

After entering the CAS number, the software presents the structural formula and information stored in the database. The classification includes structural information, physical and chemical properties, environmental fate and transmission, environmental toxicity, and human health hazards. Clicking on the symbol before classification can expand the tree structure. If the hazard endpoint data to be reviewed is insufficient, it is needed to make calculation as an alternative method. You can click on a “Data Gap Filling” button and select the desired hazard endpoint. For example, if it is desired to review the aquatic toxicity endpoint under the environmental toxicity, the QSAR button is clicked in the red box after selection, as shown in FIG. 6. After that, it is still necessary to select the QSAR model that is applicable for the selected hazard endpoint, and evaluate its applicable species, exposure pathways and predictable applicability domain. For example, if it is desired to make acute toxicity prediction under the human health hazard classification, QSAR model selection is initiated, the OECD QSAR Toolbox lists models related to the built-in database and target chemicals, as shown in Table 3. If it is desired to search an exposure pathway that is “oral” and the target chemicals meet the applicability domain, the QSAR model with a number of 2 or 6 can be selected for prediction.

TABLE 3
Demonstration for OECD QSAR Toolbox QSAR model selection

| | | | | Appli- | | |
|---|---|---|---|---|---|---|
| Num- | | | | cability | End- | Exposure |
| ber | Species | Prediction value | | domain | point | pathway |
| 1 | Mouse | 500 | mg/kgbdwt/d | Yes | LD₅₀ | Intraperitoneal |
| | | | | | | injection |
| 2 | Mouse | No prediction | | No | LD50 | Intravenous |
| | | | | | | injection |
| 3 | Mouse | 2.2E3 | mg/kgbdwt/d | Yes | LD₅₀ | Oral |
| 4 | Mouse | 1.7E3 | mg/kgbdwt/d | Yes | LD₅₀ | Subcutaneous |
| | | | | | | tissue |
| | | | | | | absorption |
| 5 | Rat | 690 | mg/kgbdwt/d | Yes | LD₅₀ | Intraperitoneal |
| | | | | | | injection |
| 6 | Rat | 2.2E3 | mg/kgbdwt/d | Yes | LD₅₀ | Oral |

The computational toxicology is one of the emerging research fields in the 21st century, which is used to study the toxicity of chemical substances. Even now, many chemicals still have no complete toxicological data. The computational toxicology can be used for preliminarily screening chemicals and hazard ranking to plan further toxicological experiment tests and understand the toxicity mechanisms of chemicals.

The Integrated Chemical Environment (ICE) is a platform released in March 2017 by the National Toxicology Program's Alternative Toxicology Methods Cross departmental Evaluation Center in the United States, which provides chemical data from animal and non-animal testing for querying test endpoints described in the chemical safety regulations and toxicology information, including acute oral toxicity, skin and eye irritation, skin sensitization and endocrine activity. In addition, ICE also provides the query and prediction of physical and chemical property data of chemicals (including solubility, melting point and molecular weight), as well as high throughput screening (HTS) data for TOX21. The expert prediction system OncoLogic for evaluating the potential carcinogenicity of chemical substances is a study and knowledge regulation set for animal or human cancers caused by chemical substances, which stimulates human experts to make predictive judgment. The user provides chemical drug information to OncoLogic™, and the potential carcinogenicity of chemical drugs is evaluated by utilizing the knowledge rules integrated in the prediction system. Open Structure-activity/property Relationship App (OPERA) also provides a QSAR/QSPR model that is reliable and meets supervision requirements for analyzing the characteristics of chemicals in the environment, including the Integrated Chemical Environment (ICE) that is released in March 2017 by the National Toxicology Program's Alternative Toxicology Methods Cross departmental Evaluation Center in the United States, provides chemical data from animal and non-animal testing for querying the testing endpoints described in chemical safety regulations and toxicology information, including acute oral toxicity, skin and eye irritation, skin sensitization and endocrine activity. In addition, ICE also provides the query and prediction of physical and chemical property data (including solubility, melting point and molecular weight) of chemicals, as well as high throughput screening (HTS) data for TOX21. The expert prediction system OncoLogic for evaluating the potential carcinogenicity of chemical substances is a study and knowledge regulation set for animal or human cancers caused by chemical substances, which stimulates human experts to make predictive judgment. The user provides chemical drug information to OncoLogic™, and the potential carcinogenicity of chemical drugs is evaluated by utilizing the knowledge rules integrated in the prediction system. Open Structure-activity/property Relationship App (OPERA) also provides a QSAR/QSPR model that is reliable and meets supervision requirements for analyzing the characteristics of chemicals in the environment, including prediction of estrogen/androgen activity, physical properties, acute toxicity, pharmacokinetic parameters, ecological toxicity parameters, etc. PRED-SKIN and other skin sensitivity data (SkinSensDB), Tox21BodyMap, and Toxic Concern Threshold (TTC) can also be used as references for predicting estrogen/androgen activity, physical properties, acute toxicity, pharmacokinetic parameters, ecological toxicity parameters, etc. PRED-SKIN and other skin sensitivity data (SkinSensDB), Tox21BodyMap, and Toxic Concern Threshold (TTC) can also be used as references.

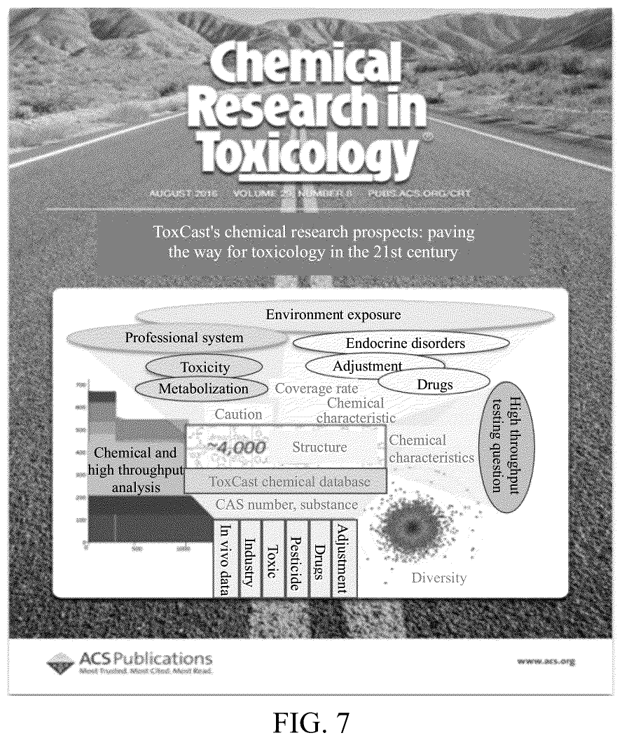

In addition, other well-known databases, such as ChEMBL (a chemical database of biologically active molecules with drug-like characteristics), have also been integrated for the characteristics of chemicals, which provide information on biologically active molecules capable of inducing drugs, and the used substance specification for Apparel and Footwear International RSL Management (AFIRM) for product packaging ingredients. The Comprehensive Global Database of per/polyfluoroalkyl substances (PFASs) functions as integrating information on per/polyfluoroalkyl substances from 15 countries or territories including the United States and the European Union, such as physical and chemical properties, uses, exposure methods and potential health and environmental impacts, so as to provide comprehensive data for policy formulation and management use. The European Commission's Priority List can provide information on whether chemicals are included in the list of “suspected endocrine disruptors”. Perkins and Will's Precautionary List clears up a precautionary list mainly including substances that have been classified by regulatory authorities as being harmful to humans or environments, listing their GreenScreen Benchmark Score and GSPI Six Classes. The Material Declaration for Products of and for the Electrical Industry comes from the International Electrotechnical Commission (called IEC for short). The database specifies that which substances, substance groups and material categories in the electrical and electronic industry need to be included in the material declaration, and provides data format specifications for software developers to exchange material declaration data. Also, toxicological databases can be used to identify potential chemical substances that may cause harm to the environment. Various toxicology databases have been established internationally, such as the US Environmental Protection Agency's High throughput Screening Toxicity Test database (ToxCast) and the Overall Endotoxicity Test Results Database (ToxRefDB). ToxCast has approximately 1800 widely sourced chemical substance data, including industrial and consumer goods, food additives, and potential green chemicals that may be safer alternatives to the existing chemicals. These data can be used for exploring the chemical and biological diffusion activity space of broad toxicological results related to regulatory concerns to generate high throughput screening data to screen chemical substances in over 700 high throughput analysis endpoints, and develop toxicity prediction models by using the chemical substances, as shown in FIG. 7. Another database, the Toxicity Reference Database (ToxRefDB), is established based on over 5000 in vivo toxicity research information, mainly refers to the guidelines or specifications of the United States Environmental Protection Agency and the National Toxicology Program, and serves as a public resource for training and validating prediction models. ToxRefDB contains mammalian toxicity information and can be used as a basis for pesticide risk evaluation, when combined with other information sources (such as exposure and metabolism). In addition, there are other publicly available ecotoxicity databases, such as ECOTOX, which contains acute and chronic toxicity values, and the data is collected from literatures; TSCATS is related to toxicity researches; HPV data, SIDS data, and Hazardous Substances Data Bank can also be used as reference.

In addition to integrating the existing chemical databases, the operational process disclosed in the present disclosure can also refer to two international indicator sources for chemical evaluation and screening: the GreenScreen® for Safety Chemicals and the Globally Harmonized System of Classification and Labeling of Chemicals (GHS) issued by the United Nations. These two international standards are used as screening conditions so as to input a chemical list and give scores to facilitate the more precise grade classification of chemicals. GreenScreen® for Safer Chemicals is a chemical safety evaluation method adopted by multinational corporations and multiple US state governments to assist in identifying chemicals of very high concern and selecting safer alternatives. Its evaluation method can be used for depth evaluation of products, processes or any chemical substances so as to determine and compare their harmfulness, and assist enterprises in selecting safer alternatives and make management decisions.

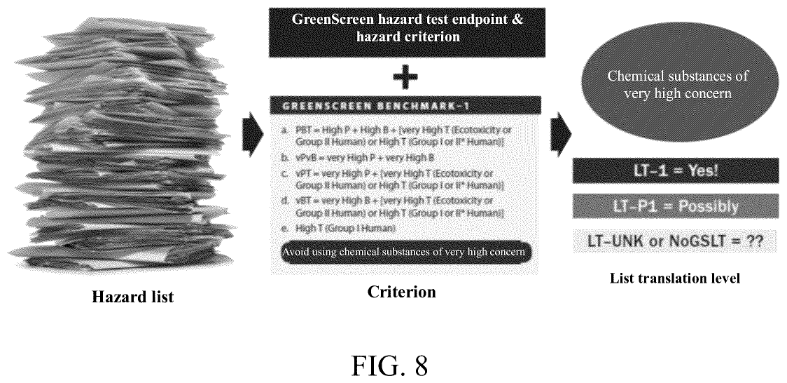

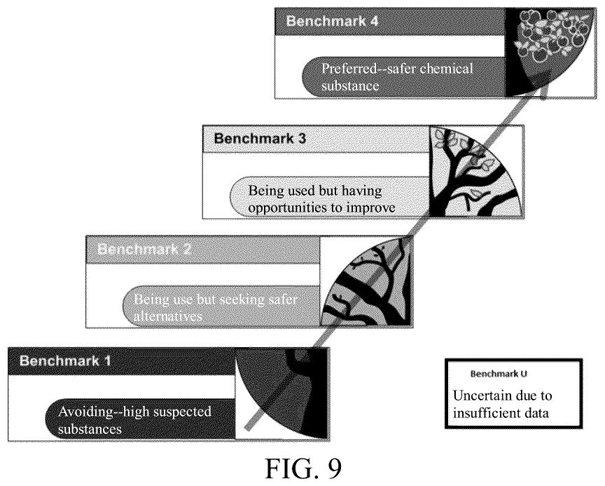

Another tool is referred to as GreenScreen® List Translator. The list translator is a list-based hazard screening method which is intended to help users quickly identify known and established chemicals receiving high concerns, and can serve as a reference template for this project (GreenScreen, 2013). As shown in FIG. 8, the GreenScreen® List can evaluate chemicals based on information from over 40 hazard lists, and then “convert” the information into a score to indicate whether a given chemical is a “safer chemical” or “chemical receiving high concerns” defined by GreenScreen®. FIG. 8 can further refer to “Clean Production Action, 2021”. The lists, jointly developed and used by authoritative scientific institutions convened by international, national, and state government agencies, intergovernmental organizations and non-governmental organizations, are collectively referred to as “GreenScreen® Specified Lists”. The chemicals are evaluated by using these GreenScreens® Specified Lists and GreenScreen® Hazard standards to assign hazard levels to relevant hazards. FIG. 9 further shows the GreenScreen® benchmark level, which is a more forward-looking development for hazard evaluation; FIG. 9 can further refer to a “website of the Ministry of Environment's Environmental Protection Personnel Training Institute”.

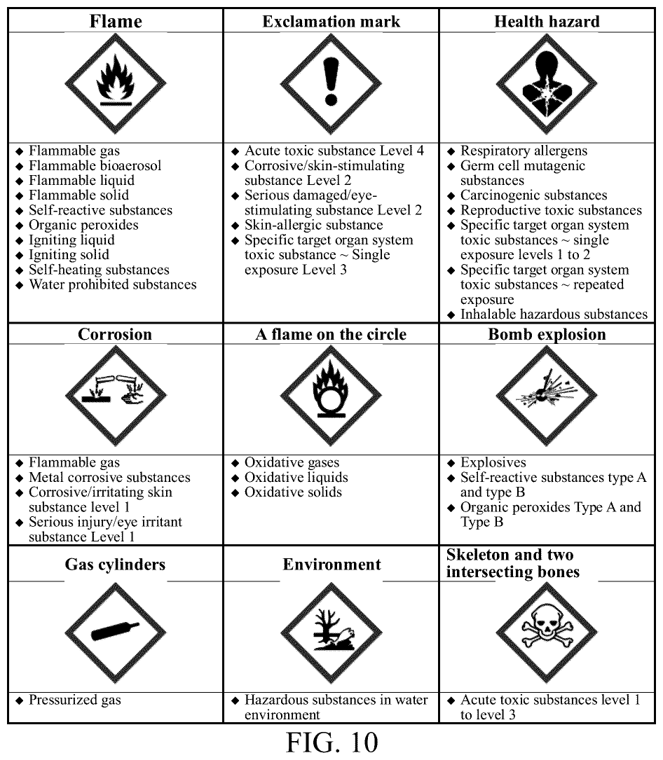

The Globally Harmonized System of Classification and Labeling of Chemicals (GHS) is an internationally recognized system for classification and labeling of chemicals. The Globally Unified System is established by the United Nations, which adopts a set of globally unified and standardized chemical classification and labeling standards to replace their own classification and labeling standards individually used by each country (Winder et al., 2005). The requirements of GHS outline the following standardization requirements: classification of chemical substances and mixtures is carried out according to physical, health, and environmental hazards, as shown in FIG. 10. FIG. 10 can further refer to “the website of the Environmental Safety and Health Center of Fu Jen University”.

One of the objectives of the present disclosure is to collect predictive analysis tools and databases from various international sources (including Europe, the United States, Japan, China and New Zealand). In the present disclosure, the existing regulations and evaluation standards are integrated in combination with bioinformatics, computer simulation (in silico) modeling, systems biology methods and the like to predict health and safety effects of chemicals on biological systems or environments. One of the objectives of the present disclosure is to promote the development of predictive toxicology as an alternative method for managing related issues. In the present disclosure, the guidelines and suggestions established by previous domestic research projects can be continued, and domestically used by creating the most suitable and excellent databases. Since the domestic safety alternative integration platform and database has not yet been perfect, one of the objectives of the present disclosure is to determine and position safe alternatives for chemical substances controlled in Taiwan, and establish a prototype of a complete database of chemical hazards. It will also integrate toxicity and concern chemical registration and reporting systems and cross departmental chemical management information collection platforms (such as the Ministry of Environment's Chemical Cloud) to reduce environmental hazards and enhance industrial values.

The present disclosure can comprise the following contents:
1. International chemical databases and chemical information are archived and integrated to obtain chemical structures and characteristic information;
2. By the chemical structure information, chemical molecular fingerprint groups are established, carcinogenicity is selected as the hazard endpoint, and a chemical characteristic prediction training set is established from a work database;
3. Per/polyfluoroalkyl substances are selected as substances to be operated; and
4. An instrument measurement and an identification flow are established.

In one embodiment of the present disclosure, the NCBI PubChem United Nations GHS database data are used. In one embodiment of the present disclosure, the Python programming language in combination with the BeautifulSoup package and the Ruby programming language in combination with the Wombat package are used to be stored in the work data base so as to obtain the latest GHS project list. In addition to the PubChem United Nations GHS database, the present disclosure can further analyze: (1) the PubChem database of the National Institutes of Health in the United States, (2) the CompTox Chemical Dashboard of the United States Environmental Protection Agency, (3) the Domestic Substances List (DSL) of Canada, (4) the ECHA announcement of the European Chemical Agency, (5) the German Federal Environment Agency's “System Regulations for the Treatment of Substances Hazardous to Water” (AwSV) (published in the Federal Legal Gazette, 2017), (6) the ChEMBL database, and (7) the AFIRM Packaging restricted substance list, (8) Fluoroalkyl polyolefin database and list, (9) European Commission's Priority List, so as to obtain information and toxicity data of chemical substances, in order to act on international convention.

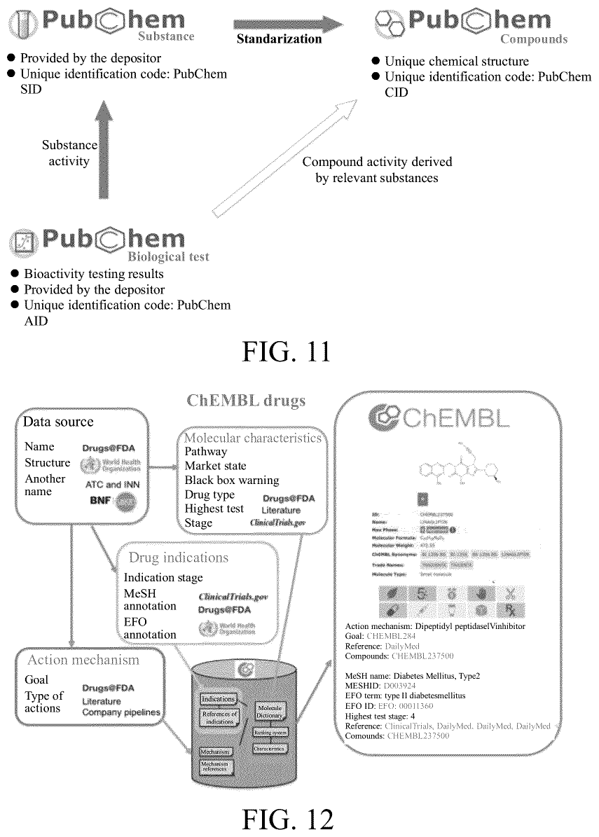

The chemical database “PubChem” is a chemical molecule database maintained by the National Center for Biotechnology Information (NCBI) of the National Institutes of Health (NIH) in the United States, and launched in 2004. PubChem can provide characteristic data of natural or artificially synthesized chemical molecules. PubChem includes three sub-databases, namely Compounds, Substance, and Bioassays. The relationship among these three sub-databases can be seen in FIG. 11, and further refers to the website of the PubChem database. PubChem covers over 100 million compounds, 300 million substances, and 1.5 million biometric data. The compound sub-database mainly stores chemical and physical properties such as the structures, chemical safety, molecular weight and the like of compounds. The substance sub-database provides information on a boiling point, a melting point, a pH value and the like of a single substance. The bioassay sub-database mainly focuses on the effects (biological activity, toxicity and the like of the molecule) of drugs or chemical molecules on organisms. The PubChem database can also be used for calculating possible stereo-structures of compounds in the database to further help users understand the structure and function of the compound.

CompTox Chemical Dashboard is a Toxic Substances Emissions Inventory (TRI) database constructed by the United States Environmental Protection Agency. The Toxic Substances Emission Inventory Database provides information on over 870,000 chemicals, including their physical properties, environmental fate, hazard assessment, human absorption, distribution, metabolism, elimination, and exposure, as well as information on structurally similar chemicals, aliases, and related literatures. CompTox can therefore gather myriad chemical toxicology information.

For the Domestic Substances List (DSL) of Canada, the chemical substances on this list are subject to commercial government regulation when being manufactured and imported in Canada. The initial version list of DSL was first published in Canada Gazette, Part II on May 4, 1994. In the initial version list, approximately 23,000 chemicals are regulated and 12 times are updated a year on average, with addition or removal of chemical substances. The latest Excel file of the chemical substance list in this list can be downloaded from a database in an official website. Besides, when the DSL is stipulated in Canada, the existence, bioaccumulation, environmental and human hazards of various chemical substances are evaluated, and these toxicity information is important for chemical testing.

The ECHA announcement of the European Chemical Agency's Chemicals Administration is based on: (1) Annex 17 of the REACH regulation, (2) Candidate List of Substances of Very High Concern, and (3) List of Substances of High Concern-Priority List. The Annex 17 of the REACH regulation refers to a restriction list issued under the European Union's Chemical Registration, Evaluation, Authorization and Restriction Act (REACH), which lists the occasions and conditions where the use of chemical substances is prohibited and will be updated periodically. The Candidate List of Substances of Very High Concern refers to Chapter 59 (10) of the European Union's Chemical Registration, Evaluation, Authorization and Restriction Act (REACH); the use of substances on the list in the European Union is subject to regulation. The List of Substances of Very High Concern—the Priority List is issued by the European Chemical Agency, concerning that in REACH, it is required for the European Chemicals Agency (ECHA) to recommend priority substances listed in REACH from the candidate list, and submit transitional arrangements and related exemptions and review periods of these substances to the European Commission while taking into account the opinions of member state committees. The list of Substances of very high concern-Priority List includes all substances already included in the ECHA draft or final recommendation, and substances that are not recommended will be re-evaluated in subsequent rounds.

For the German Federal Environment Agency's System Regulation for the Treatment of Hazardous Substances in Water (AwSV), it aims to protect a water body from adverse changes caused by chemicals and mixtures. The German Federal Environment Agency requires that substances and mixtures processed or stored in facilities located in Germany are classified into three water hazard levels (WGK) according to their water hazard characteristics: (1) WGK1: slight harm to water; (2) WGK2: clearly harm to water; and (3) WGK3: highly harm to water. In addition to the three water hazard levels, substances can also be classified as no harm to water (nwg) or general harm to water (awg). There are approximately 12,000 pieces of chemical information in this list.

The ChEMBL database provides information on biologically active molecules capable of inducing drugs. The ChEMBL database is currently managed and maintained by the European Institute for Biological Information of the European Molecular Biology Laboratory (EMBL-EBI) in the UK, covering over 2.3 million compounds and 1900 activity analysis data. The data sources of the ChEMBL database include published literature, clinical data and other publicly available databases, etc. The ChEMBL database provides users with information on the molecular characteristics, drug induction, drug action mechanisms and clinical data of compounds and their metabolic mechanisms involved in biological reactions. The ChEMBL database can effectively provide users with basic information on the molecule and its reactions as a drug. The ChEMBL database can be seen in FIG. 12 and further refer to the website of the ChEMBL database.

The International Organization for the Management of Restricted Chemical Substances (AFIRM Group) has proposed substance specifications for the use of product packaging ingredients. The members of the organization include many famous clothing brands such as Adidas and H&M. The members must conduct testing and reporting on the ingredients listed on the restricted substance list. The information on limited values, uses, testing methods and limited values required to be reported for the restricted substances in various types of packaging is listed in the list of restricted substances for packaging, and provided to member brands of the organization as a basis for production, testing and acceptance of testing reports. In addition, a restricted substance list has also been proposed for the product itself, which is used for specifying the ingredients used in production of clothing, shoes and socks, accessories, jewelry, sports equipment, wearable equipment, household textiles and the like by manufacturers. Information on the limit values, uses, testing methods and limit values that need to be reported for restricted substances are also proposed to monitor member brands.

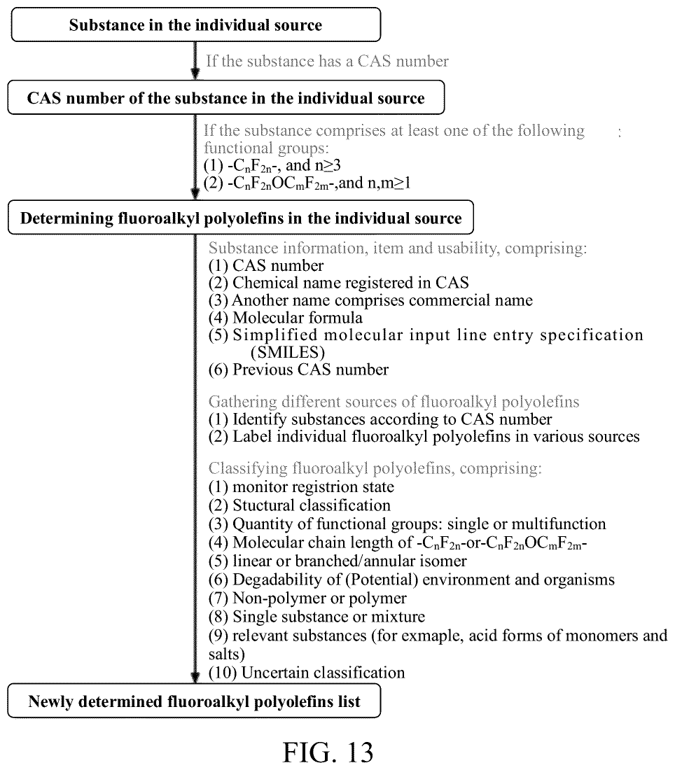

Comprehensive Global Database of PFASs is maintained by the PFC group of the Organization for Economic Cooperation and Development (OECD) and the United Nations Environment Programme (UNEP), and was established in 2012. Per/polyfluoroalkyl substances are a type of chemical substances that includes perfluorinated alkyl polyolefins (PFCs) and perfluorinated fatty acid salts (PFAAs). Such the chemical substances began to be widely used in the 1940s. Due to the characteristics such as water resistance, oil resistance and low friction, such the chemical substances are in most cases used as surface coatings for food packaging, containers, etc. However, since such the chemical substances are high in stability and difficultly decomposed in the environment, their use is monitored by the United States, the European Union and other countries in recent years. The Comprehensive Global Database of PFASs integrates control information on per/polyfluoroalkyl substances from 15 countries or territories such as the United States and the European Union, for example physical and chemical properties, uses, exposure methods, potential health and environmental impacts, so as to provide comprehensive data for policy formulation and management. In addition, the database also includes relevant research findings and suggestions, including management policies and regulations, clearance methods and available alternatives in various countries, to help policy makers make decisions. The screening flowchart for the updated list of the Comprehensive Global Database of PFASs is seen in FIG. 13, and can further refer to the website of the Comprehensive Global Database of PFASs. In addition, the highly credible Comprehensive Global Database of PFASs also includes the PFAS Master List of PFAS Substances from the US Environmental Protection Agency (EPA), as well as the Suspect List of Possible Per/Polyfluoroalkyl Substances (PFAS) provided by the US National Institute of Standards and Technology (NIST). The present disclosure has collected approximately 15,000 per/polyfluoroalkyl substances from the above-mentioned three sources (see Table 4). The present disclosure also collects, organizes and adds numbers of other database systems except CAS numbers, for example Compound Identifier (CID) in the PubChem database system. Meanwhile, the Simplified Molecular Input Line Entry Specification (SMILES) that is missing from the data source is also supplemented.

TABLE 4
Database and list of collected per/polyfluoroalkyl substances

| | Maintenance | Quan- |
|---|---|---|
| Database name | organization | tity |
| Comprehensive Global Database of | Organization for | 4730 |
| PFASs | Economic | |
| Comprehensive Global Database of | Cooperation and | |
| PFASs | Development (OECD) | |
| Total List of Per/polyfluoroalkyl | The U.S. Environmental | 12034 |
| Substances | Protection Agency | |
| (PFAS Master List of PFAS | (USEPA) | |
| Substances) | | |
| Suspect List of Possible | The U.S. National | 4969 |
| Per/Polyfluoroalkyl Substances | Institute of | |
| (PFAS) | Standards and | |
| (Suspect List of Possible Per-and | Technology | |
| Polyfluoroalkyl Substances (PFAS)) | (USNIST) | |

SMILES is a string encoding system for describing the structures of chemicals, which can be converted into the three-dimensional structures of chemicals and is widely used in the field of computational chemistry. Therefore, numbers of other database systems are collected and organized in the present disclosure, including CAS numbers, such as CID in the PubChem database system. Meanwhile, the SMILES that is missing from partial data sources is also supplemented to provide comprehensive chemical information.

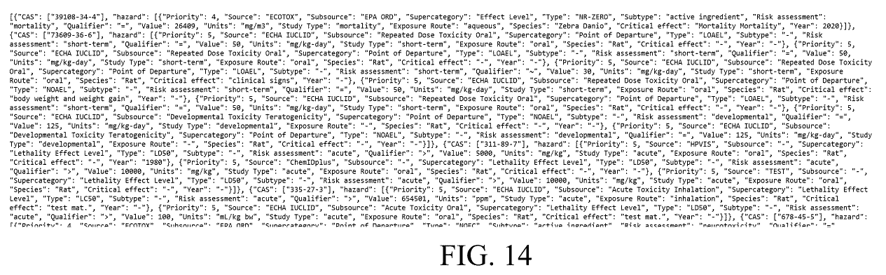

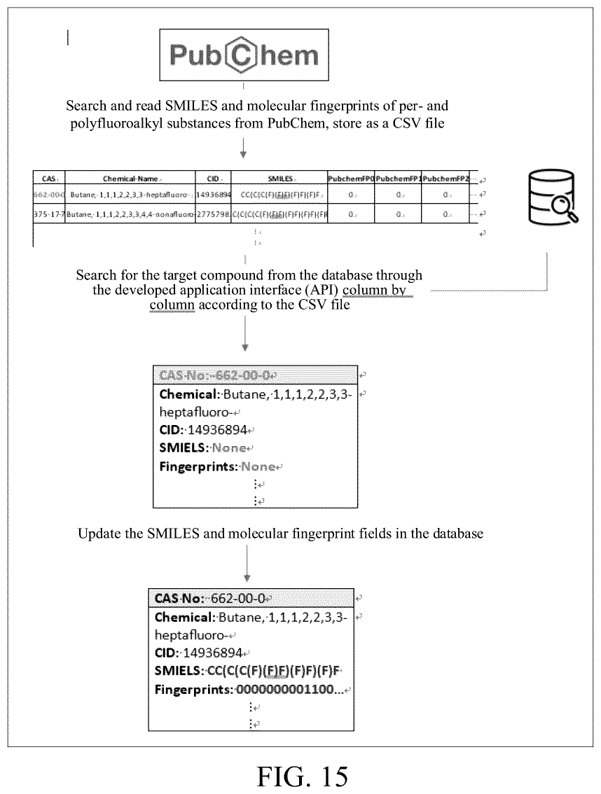

The CompTox database is a valuable resource which is provided by the US EPA and has extensively collected and organized toxicological information of chemical substances. This database focuses on providing toxicological experimental data for various chemical substances, which is crucial for understanding their harmful characteristics and potential risks to human health and the environment. Toxicological experimental data is a result for studying the adverse effects of chemicals in living organisms through a series of experimental methods. These data cover multiple aspects, including acute toxicity, chronic toxicity, mutagenicity, teratogenicity, etc. These experiments can reveal the degree of damage that chemicals may cause to cells, tissues and the entire biological system. The toxicology experimental data in the CompTox database are not only numbers, but also include detailed descriptions of experimental methods, setting of experimental parameters, statistical analysis of experimental results, etc. This enables researchers to better evaluate the reliability and applicability of data to ensure the correct understanding and application of toxicological information of chemical substances In addition, one of the advantages of the CompTox database is its integrated features. It not only integrates internal experimental data from the US Environmental Protection Agency, but also includes third-party data from different sources, which expands the scope and richness of the database. Therefore, in the present disclosure, toxicological experiment data of PFAS, with a total of 5120 records, was also supplemented by the CompTox database. FIG. 14 shows that 5120 toxicological experiment data of PFAS are stored in the CompTox database in JSON format. FIG. 15 shows a process for establishing SIMILES and molecular fingerprint database for per/polyfluoroalkyl substances.

The Priority List of the European Commission is described as follow. To establish standards and priorities for listing chemicals on the list of suspected endocrine disruptors, the European Commission began a series of studies in 2000 AD to establish a Priority List through a coherent method to further evaluate their roles in endocrine disruptors. The Priority List is established in two stages. Firstly, independent review of evidence of endocrine disrupting effects and human or animal exposure is conducted, followed by consultation with stakeholders and committee scientific members to determine the priority of implementation matters. The European Commission analyzed 564 suspected endocrine disruptors published by various organizations, and ultimately classified 194 first class chemicals and 126 second class chemicals, which were compiled into a database archive. The review process of substances includes:
(1) A chemical work list is complied from a “suspected endocrine disruptors” list published by various organizations, and reports and papers that involve the endocrine disruptors of specific chemicals are found in combination with scientific literature search. In order to ensure the comprehensiveness of the list as much as possible, the draft list will be discussed in meetings with major stakeholders (including representatives from government, industry, and non-governmental organizations). Data of the endocrine effects of chemicals on humans, other vertebrates and invertebrates will be collected and included in the database. In addition, if there is any persistence of each chemical in the environment and the possibility of accumulation in organisms exposed to that environment (i.e., bioaccumulation), it will also be collected together.
(2) The existing information is reviewed to find chemicals that may have high persistence in the environment (i.e., decomposition resistance) or be produced in large quantities by the industry (i.e., over 1000 tons per year). The two standards are adopted because humans and animals have a higher chance of being exposed to these chemicals under these two conditions, thus posing a greater potential risk of harm.
(3) Based on expert advices, the information of the high persistence or mass production chemicals identified in step two is reviewed to determine the strength of evidence for endocrine disruption, and categorize the chemicals into one of the following three categories.
Category 1: Evidence of endocrine disrupting activity on at least one complete animal species;
Category 2: at least some biological activity evidence related to in vitro endocrine disruption;
Category 3a: No evidence of endocrine disruption activity;
Category 3b: Insufficient data.
(4) The existing information on chemicals classified as Class I in Step 3 is reviewed to determine the likelihood of actual exposure to humans or wildlife. The highest attention is paid to chemicals into which humans or wildlife are expected to be exposed; moderate attention is paid to chemicals in which humans are not expected to be exposed to but wild animals may be exposed; minimum attention is paid to chemicals into which humans or wildlife is not exposed.

According to GreenScreen® Method, after the chemical substance analysis reports from all source databases are downloaded, a program is used to convert and store the information in JSON format for data exchange. Subsequently, a program is written to calculate GreenScreen® hazards based on a scoring method to evaluate and integrate 18 levels of hazards to calculate comprehensive risk evaluation for future query.

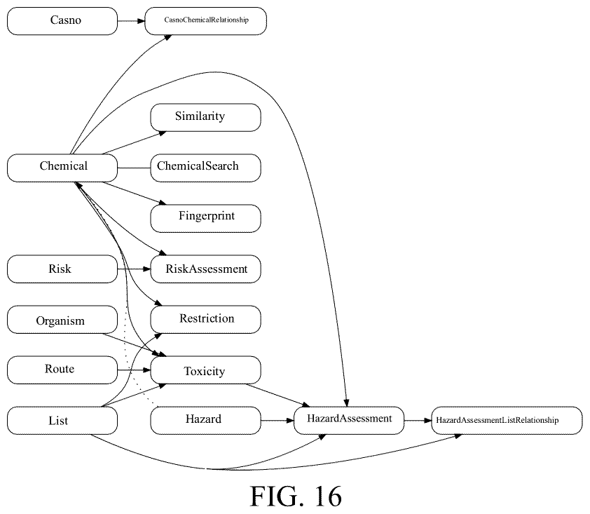

The database design of the present disclosure can include 16 tables, including CAS numbers, chemical names, relationship tables between CAS numbers and chemical names, hazards, hazard evaluation, hazard evaluation and restriction list relationship indexes, restricted chemicals, toxicity, risk, risk evaluation, exposure pathways, restriction lists and species summary table. These tables also include relevant information between restricted specifications and tables in different countries and regions. 16 tables consist of 14 primary tables and 2 auxiliary tables for storing many-to-many relationships. The auxiliary tables store indexes between primary tables, and multiple-to-many relationship information including relationships between CAS numbers and chemicals and between regulatory lists and hazard evaluation. A database model association diagram is shown in FIG. 16, where chemicals are directly associated with toxicity, restrictions, hazard evaluation and risk evaluation. In the future, searching for chemicals will allow for the retrieval of relevant toxicity data, regulatory restrictions in various countries, as well as hazard and risk evaluation levels.

The work database of the present disclosure is constructed by using a Docker container system, and provides the characteristics that the database is easy to manage, maintain and transfer. The system adopts a Docker Compose to cascade a database management system (MariaDB), a database website (Ruby on Rails), a caching system (Redis) and an automatic updating workflow system (Airflow). It is planned to adopt the open source Airflow workflow language dominated and maintained by the Apache Foundation to compile and automatically update the workflow. Airflow is developed in Python language, which is a commonly used framework in the field of data science and big data. Both Google and Amazon cloud computing provide Airflow services to cascade their own cloud services. Domestic companies such as Cathay Financial automatically update data through Airflow, so as to reduce maintenance manpower and time costs. The present disclosure can further include 15 database update program codes, and an Airflow container is added to Docker Compose for the convenience of unified management, initiation of updates and storage in the database.

The supplemented chemical information includes SMILES information. In the present disclosure, SMILES is converted into PubChem molecular fingerprint groups by using PaDEL descriptor software and stored in the work database. In order to conveniently and quickly obtain chemical characteristic data from the work database, the inventor of the present disclosure developed a RESTful (Representative State Transfer) application programming interface (API), by which CAS numbers or PubChem CID can be utilized to search hazard information of specific chemicals. The endpoint of the obtained data is as follows: GET/chemicals/(parameter 1: casno or pubchem_cid)/(parameter 2: corresponding database code)/(parameter 3: all). Parameter 1-parameter 2 are described as follows:
(1) Parameter 1: a database expected for query data, such as CAS number (input casno) or PubChem CID (input pubchem_cid).
(2) Parameter 2: a corresponding data number, for example, input 335-67-1, and it represents CAS No. 335-67-1, i.e., perfluoooctanoic acid (PFOA).
(3) Parameter 3: fill in all hazard endpoints (input all), or human chronic toxicity (g1_human), human acute toxicity (g2_human), environmental toxicity (ecotox), environmental fate (fate), and physical hazards (physical).

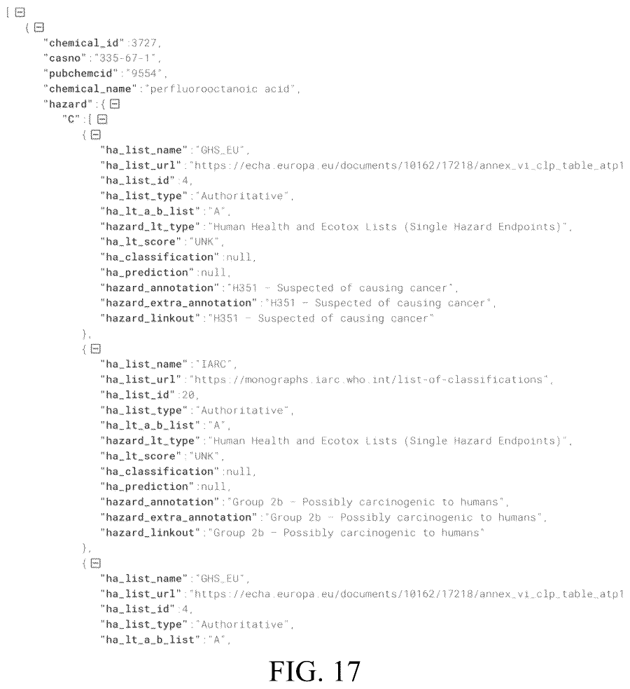

The format of the returned file is JSON (JavaScript Object Notation), which includes: hazard endpoints, data types (2 hazard evaluation methods), data sources, credibility, hazard evaluation levels or predicted values, annotations. FIG. 17 shows partial data returned from the endpoint query. Where, “chemical_id” represents the identification code of this compound in the database; “Casno” represents its CAS number; “pubchemcid” represents its PubChem CID; “hazard” represents its hazard information. “hazard” contains more detailed hazard categories. FIG. 17 shows several data in carcinogenicity, “C” is called carcinogenicity for short. And under each subcategory of hazards, information from all data sources is included:
(1) “ha_list_name” is a name of a data source;
(2) “ha_list_url” is a website of a data source;
(3) “ha_list_type” is a type of data;
(4) “ha_lt_a_b_list” is credibility;
(5) “ha_lt_score” is one of hazard evaluation scores;
(6) “ha.classification” is a hazard evaluation level; and
(7) “hazard_annotation”, “hazard_extra_annotation” and “hazard_linkout” are annotations and additional connections.

The example shown in FIG. 17 presents toxicity information of data from EU GHS and International Agency for Research on Cancer. FIG. 17 shows data presentation of chemical substance information and hazard data inquired from the work database.

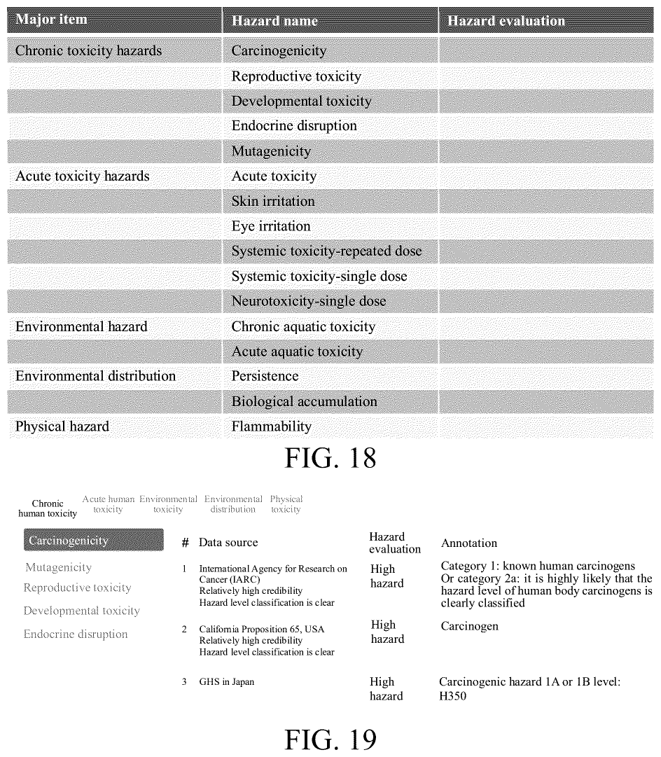

In order to make users more concisely and clearly understand inquired information, a chemical hazard information interface and toxicity data visualization presentation portion is also designed in the present disclosure. FIG. 18 and FIG. 19 show design logic and visual display examples disclosed in the present disclosure. FIG. 18 shows a data presentation interface design disclosed in this disclosure, which divides hazards into five major categories and subdivides hazard names; FIG. 19 illustrates a data presentation interface design disclosed in the present disclosure, which can include five major items and subdivided hazard names. The five major categories of hazards can be switched with the top of the table as a tab. The names of the hazards subdivided in the five major categories of hazards are placed at the left side of the table and presented as labels. After the specified label is clicked, the table is guided to a corresponding target, and shows its related data.

In the present disclosure, all data sources are integrated and stored in the work database based on the list, the hazard endpoints are used as hazard evaluation information of carcinogenicity, the hazard evaluation of data is then transformed into hazard levels by using the GreenScreen Method. The hazard evaluation of data sources related to carcinogenicity are exported from the work database, high-credibility lists (such as lists provided by multinational organizations, the United States and the European Union) are retained, hazard evaluation information with unknown hazard levels is removed, and finally 3810 pieces of hazard information are obtained. Since the GreenScreen method includes a list translator level (list translator score) and a hazard evaluation level (Benchmark score), classifications are comprehensively labeled (high hazard H, medium hazard M, low hazard L) based on combination of hazard translator level (level 1, possible level 1) and hazard evaluation level (H, M, L) in the present disclosure. If the hazard translator level is “level 1”, the classification is labeled as high hazard H. If the hazard translation level is “possible level 1”, the classification is labeled as medium hazard M. The hazard evaluation levels H, M, L are transformed into corresponding classifications that are labeled as high hazard H, medium hazard M, and low hazard L. Table 5 is a data field table according to an embodiment of the present disclosure.

TABLE 5
Data field table of training dataset for
chemical characteristic prediction

| Domain name | Field description |
|---|---|
| Hazard evaluation | Hazard evaluation number in |
| number | work database |
| Chemical number | Chemical number in work database |
| Chemical name | Common chemical name |
| Molecular number CID | PubChem Compound ID |
| of PubChem database | |
| Data source | Data sources of hazard evaluation |
| | information |
| Credibility | List credibility defined by GreenScreen |
| | Method |
| Hazard's definition is | Hazard's definition defined by |
| clear or not | GreenScreen Method is clear or not |
| Hazard translationlevel | List translator score defined by |
| | GreenScreen Method |
| Hazard evaluation level | Benchmark score defined by GreenScreen |
| | Method |
| Classification label | Classification label of the present disclosure |

The QSAR model should provide the following five pieces of information according to the decision made by Organisation for Economic Cooperation and Development (OECD) in November 2004:
1. Prediction endpoint with complete definition
2. Explicit algorithm
3. Predicable applicability domain with explicit definition
4. Prediction accuracy degree is tested by using a proper method
5. Explanation of a prediction mechanism is provided if feasible

The present disclosure adopts the principle provided by OECD to design a QSAR model and predict the classification label of the target hazard endpoint through the QSAR model. In the present disclosure, “carcinogenicity” is used as the target hazard endpoint. The construction method is described in detail below.

### Prediction of Endpoints

The prediction endpoint of the QSAR model described in the present disclosure is “carcinogenicity”. Chemicals that may be or confirmed to have a carcinogenic risk are classified as positive; chemicals that do not have relevant hazards are classified as negative.

### QSAR Algorithm

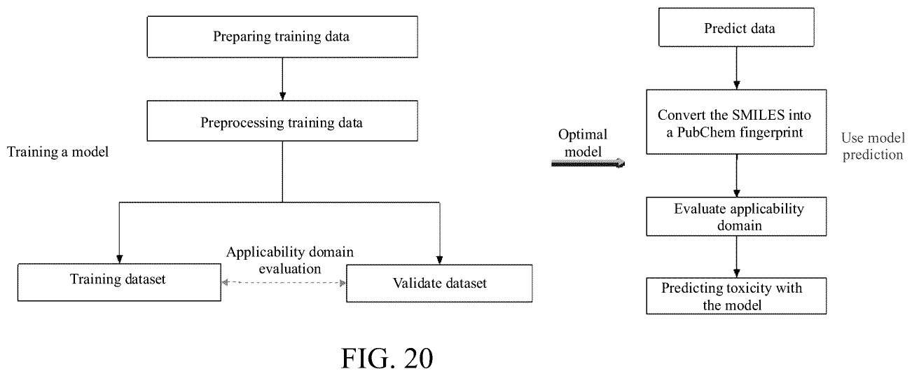

FIG. 20 shows a rough process of a QSAR algorithm. First, a batch of training data is prepared and then divide them into a training dataset and a validation dataset after preprocessing, subsequently model training is performed by using the training dataset, effect evaluation is performed through validation dataset to select an optimal model for prediction. Prediction data is first converted from a SMILES expression into a PubChem fingerprint, followed by an applicability domain evaluation to confirm that the chemical is in the applicability domain, and toxicity prediction is performed with the model. Next, each step flow will be described.

### Algorithm Flow-1. Preparation of Training Data

The training data sources may comprise the following three sources:
1. International list dataset (data quantity=3,810) collected based on GreenScreen Method. This dataset integrates chemical lists from multiple international list sources. As most of the lists are positive lists, i.e., the chemical is recorded if it has carcinogenic hazards, and therefore the chemicals are often recorded as having “high” and “medium” carcinogenic hazards, which will be recorded as positive in the QSAR.
2. FooDB (https://foodb.ca/data, quantity=70,477). This dataset is a free and publicly available food related database in which the chemical components that may appear in food are recorded. These chemical components may give these foods fragrance, color, taste, texture, aroma, etc, and therefore they are considered as negative during the establishment of the QSAR model.
3. Carcinogenicity dataset (data quantity=863) collected by DeepCare. The original sources of this dataset are the National Center for Toxicological Research liver cancer database (NCTRlcdb) and the Carcinogenic Potential Database (CPDB) of the US Food and Drug Administration. This data set excludes inorganic compounds, mixtures and organometallic compounds. This dataset includes positive and negative chemicals.

### Algorithm Flow-2. Preprocessing of Training Data

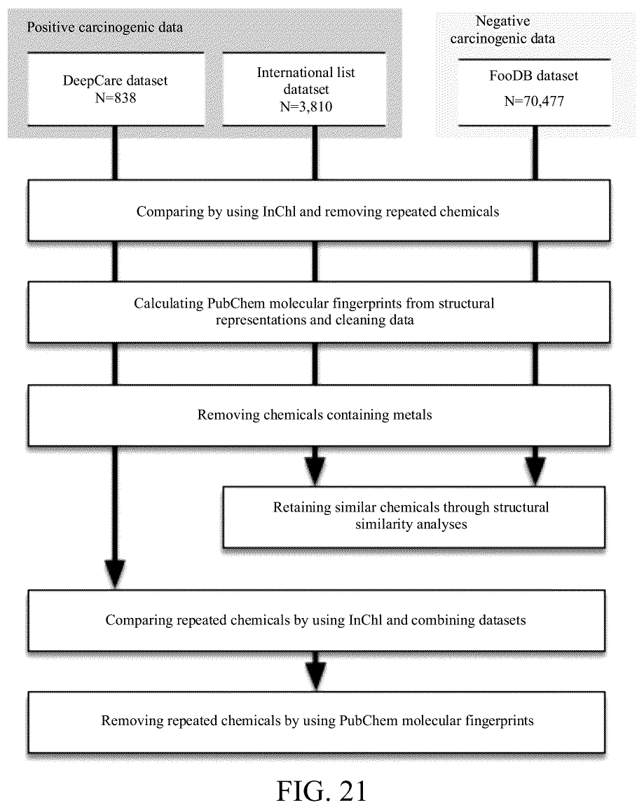

Due to different sources of the training data, the training data need to be integrated. The integration flow is as shown in FIG. 21. First, it is confirmed that each chemical data item from each source has an simplified molecular input line entry specification (SMILES) string to represent a chemical structure, and the data items that do not contain SMILES will be excluded. Subsequently, whether the dataset from the same source has repeated chemical items is confirmed by International Compound Identifier (InChI), and the repeated chemicals will be removed. The SMILES structural string of each data source is converted into a PubChem fingerprint by utilizing PaDEL, and the chemicals that cannot be converted into PubChem fingerprint will be removed.

In order to further improve the usability of the QSAR model described in the present disclosure, inorganic compounds and metal organics are excluded. Therefore, after data cleansing is performed by the PubChem molecular fingerprints, through SMILES structural string analyses, the chemicals having metal are removed from the dataset applicable for this model. The definitions of the excluded elements include: Sc, Ti, V, Cr, Mn, Fe, Co, Ni, Cu, Zn, Y, Zr, Nb, Mo, Tc, Ru, Rh, Pd, Ag, Cd, La, Ce, Pr, Nd, Pm, Sm, Eu, Gd, Tb, Dy, Ho, Er, Tm, Yb, Lu, Ac, Th, Pa, U, Np, Pu, Am, Cm, Bk, Cf, Es, Fm, Md, No, Lr, Hf, Ta, W, Re, Os, Ir, Pt, Au, Hg, Rf, Db, Sg, Bh, Hs, Mt, Ds, Rg, Cn, Al, Ga, In, TI, Sn, Pb, Bi, Si, Ge, As, Sb, Se, Te, Po, and At.

Since the FooDB dataset is used as a negative dataset source, its data quantity is much greater than that of the positive international list dataset. To reduce analysis error caused by data imbalance, the FooDB dataset is subjected to similarity comparison with the international list dataset before the combination, and retains items in the FooDB similar to those in the international list dataset.

The similarity comparison method is described as follows:
1. Chemicals in FooDB are subjected to similarity comparison with the international list dataset one by one.
2. 5 chemicals in the international list dataset that have the closest PubChem fingerprints of FooDB chemicals to the query are found.
3. The Tanimoto coefficient is calculated by PubChem fingerprint, the Tanimoto coefficients of the 5 chemicals in the international list datasets that are the closest to the query chemical are calculated one by one, and the average value of the Tanimoto coefficients is taken. Therefore, the average similarity degree of the query chemicals relative to the international list dataset can be obtained.

$$Tanimoto coefficient : {s}_{i , j} = \frac{{n}_{11}}{{n}_{11} + {n}_{01} + {n}_{10}} , {d}_{i , j} = 1 - {s}_{i , j} ( s : similarity ; d : distance )$$

n₁₁: the fingerprints of two compounds to be compared are both 1; n₀₁ and n₁₀: one of the fingerprints of two compounds to be compared is 0, and the other is 1.
4. The distance of 0.5 in the Tanimoto coefficients is set as the similarity comparison threshold, the FooDB chemicals within the distance are retained, and the rests are discarded.

After similarity comparison, datasets are combined, and overlapped chemical items are compared using InChI. If there are overlapped chemicals with conflicting labels, priority will be given to the following: international list dataset>DeepCare dataset>FooDB dataset as labels. The reasons are that the sources of the international list dataset are all carcinogenicity related lists compiled by the governments and other impartial agencies, wherein many experiments have confirmed the potential or actual carcinogenic hazards in the past. The DeepCare dataset derives from the US Food and Drug Administration and is also credible. The FooDB data is established by detecting the characteristics of the chemical in food, and the chemicals in the FooDB are considered to be less carcinogenic and can fill in negative data, so the FooDB data is listed last in order.

Finally, after the datasets are combined, repeated chemical items are removed by using PubChem molecular fingerprint to ensure that the molecular fingerprint is in one-to-one correspondence to the label.

### Algorithm Process-3. Training Dataset and Model Training Method

After being preprocessed, the training data is divided into a training dataset and a validation dataset. The training dataset will be used for definition of the predicable applicability domain and model training. The validation dataset is used for evaluating the precision of the model, and selecting the optimal model for model prediction. The division of the training and validation datasets is repeated 10 times with different division random seeds to obtain the average performance of the model training.

### Algorithm Process-4. Definition of Predicable Applicability Domain

The applicability domain of the present disclosure must meet three conditions:
1. The query chemical should not contain the above-mentioned excluded elements. If the chemical contains the element, it is not in the predicable applicability domain.
2. The query chemical should meet the molecular weight interval where the used training dataset is located; for example, if the training dataset with a molecular weight of 100 g/mol-600 g/mol is used, the chemical with a molecular weight of 100 g/mol-600 g/mol is in the predicable applicability domain.
3. The query chemical and the chemicals in the training dataset should have sufficient similarity degree, which must be at least higher than the 95th percentile similarity in the training dataset. A similarity calculation method is as follows:
   (1) First, by utilizing the Nearest Neighbors of sklearn package in python programming language, 5 chemicals in the training dataset that are closest to PubChem fingerprints of the query chemical are found.
   (2) The Tanimoto coefficient is calculated using PubChem fingerprint; the Tanimoto coefficients between the query chemical and each of the 5 closest chemicals in the training datasets are calculated, and the average value of the Tanimoto coefficients is taken to obtain the average similarity degree of the query chemical relative to the training dataset.

$$Tanimoto coefficient : {s}_{i , j} = \frac{{n}_{11}}{{n}_{11} + {n}_{01} + {n}_{10}} , {d}_{i , j} = 1 - {s}_{i , j} ( s : similarity ; d : distance )$$

n₁₁: the fingerprints of two compounds to be compared are both 1; n₀₁ and n₁₀: one of the fingerprints of two compounds to be compared is 0, and the other is 1.
(3) The average similarity degrees between each training data item and the training dataset population is calculated by the same method one by one, the similarity degree of the 95th percentile in the training dataset is found by ranking, and the 95th percentile is used as an evaluation criterion.
(4) The similarity degree of the query chemical is compared with that of the 95th percentile in the training dataset. If the similarity degree is higher than the evaluation criterion (having sufficient small distance), it is deemed as being in the predicable applicability domain; on the contrary, it is not predicable.
(5) In addition, the similarity degrees can also be calculated by Manhattan or city-block distance, which can be used for calculation of average similarity of (2). The rest flows (1)-(4) are all the same.

$$Manhattan or city - blockdistacnce : {d}_{i , j} = {\sum }_{k = 1}^{p} \vert {x}_{i , k} - {x}_{j , k} \vert ( d : distance )$$

p: the quantity of fingerprints; x_{i,k}, x_{j,k}: numerical values of compounds i and j in fingerprint k.

### Algorithm Process-5. Model Adoption and Training Method

The model of the present disclosure can be subjected to model training and prediction by using three methods such as support vector machine (SVM), C5.0 decision tree model, and Random forest.

SVM is to convert data into high dimension through kernel functions and find a hyperplane in a high-dimensional space so as to distinguish two types of different labels which are “carcinogenic” and “non-carcinogenic”. Where, SVM adopts two different kernel functions for training: a polynomial kernel (poly) and a radial basis function kernel (rbf). In the training process of SVM, hyperparameters C and γ (when the rbf kernel is used) need to be selected and adjusted, and therefore the adjustment of the hyperparameters is performed by using a 10-fold cross validation method in conjunction with random search. That is, the training dataset is divided into 10 equals, wherein one equal is used as an internal validation dataset, other nine equals are used as training data and trained one by one, and training performances are averaged to obtain the average performance under this group of hyperparameter. Then, another group of hyperparameters is generated at random. The division and model training of the above 10 equals are repeated to find optimal hyperparameter configuration.

The C5.0 decision tree model is to generate one tree according to training data and find a classification rule in the training data through divisions of the characteristics, and the classification rule is used for prediction of new data. Where, the C5.0 decision tree model still has hyperparameters to be selected—maximum decision tree depth (max depth), minimum samples leaf of leaf nodes (min samples leaf). Similarly, the 10-fold cross validation method in combination with random search is used for the hyperparameter adjustment.

The random forest is to randomly extract training data to generate multiple decision trees and perform prediction and classification through a majority decision method. The hyperparameter adjustment item of the random forest has max depth and min samples split required for internal node redistribution. Similarly, the 10-fold cross validation method in combination with random search is used for the hyperparameter adjustment.

Model evaluation pointer adopts accuracy, sensitivity, specificity, precision, negative predictive value (npv), F1-score and Matthew's correlation coefficient (mcc). The calculation formula of the evaluation index is as follows:

$$Accuracy ( accuracy ) = \frac{True positive ( TP ) + True negative ( TN )}{True positive ( TP ) + True negative ( TN ) + False positive ( FP ) + False negative ( FN )}$$

$$Sensitivity ( sensitivity ) = \frac{True positive ( TP )}{True positive ( TP ) + False negative ( FN )}$$

$$Specificity ( specificity ) = \frac{True negative ( TN )}{True negative ( TN ) + False positive ( FP )}$$

$$Precision ( precision ) = \frac{True positive ( TP )}{True positive ( TP ) + False positive ( FP )}$$

$$Negative predictive value , npv ( negative predictive value , npv ) = \frac{True negative ( TN )}{True negative ( TN ) + False negative ( FN )}$$

$$F 1 - score ( F 1 - score ) = \frac{2 \times precision ( pr ecision ) \times sensitivity ( sensitivity )}{precision ( precision ) + sensitivity ( sensitivity )}$$

$${Matthew}^{′} s correlation coefficient ( mcc ) = \frac{TP \times TN - FP \times FN}{\sqrt{( TP + FP ) ( TP + FN ) ( TN + FP ) ( TN + FN )}}$$

The present disclosure can use the above flow for model training and the flow is feasible after being confirmed by expert meeting, and then a model prototype for chemical characteristic structure prediction is established.

In one embodiment of the present disclosure, a trial operation of high-tech industrial discharge wastewater samples and PFAS chemical substances is made to establish a model prototype for characteristic structures of cosmetics. In one embodiment of the present disclosure, a trial operation of high-tech industrial discharge wastewater samples and chemical substances of per/polyfluoroalkyl substances (PFAS) is made. The model prototype is tested by a prediction operation flow, which at least comprises: PFOA (Perfluorooctanoic acid, CAS: 335-67-1), PFOS (Perfluorooctanesulfonic acid, CAS: 1763-23-1), and PFNA (Perfluorononanoic acid, CAS: 375-95-1).

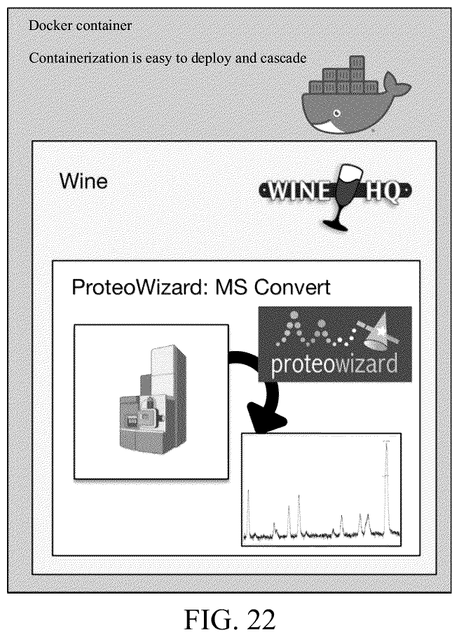

The present disclosure comprises establishing an instrument measurement and identification process cascaded with a chemical characteristic prediction operation process. The present disclosure comprises converting instrument raw data into public format by a utility program. In the present disclosure, Docker containerized programming language and a Wine framework are used to execute ProteoWizard MSConvert conversion software so as to convert instrument measurement raw data into public format mzXML. The execution method of the present disclosure comprises executing the ProteoWizard Docker image file through Docker program, executing Wine program to guide msconvert program to be executed, converting the above file into an mzXML file, and outputting the mzXML file. The schematic diagram of the execution method of the present disclosure can be shown in FIG. 22. FIG. 22 shows the conversion of raw data by MS Convert program.

The present disclosure comprises calling XCMS to perform wave peak detection. XCMS analysis software is developed by Scripps Institute, a well-known research institution in the United States, which is one of preferred analysis software in the field of small molecule and metabolite research, and has an Application Programming Interface (API) that can be used to write programs for cascading. XCMS can analyze data from a liquid chromatography mass spectrometer and a gas chromatography mass spectrometer. In addition, it can help users to process, analyze and visualize massive mass spectrometry data. Preprocessing, wave peak detection, wave peak alignment and statistical analysis and other operations can be performed on mass spectrometry data by using XCMS. When XCMS is used to perform feature extraction, different methods can be selected, for example, based on peak shape or signal intensity. Then, the extracted features are subjected to wave peak alignment to eliminate variability between different samples. Finally, analysis and visualization are performed using a statistical method.

In the present disclosure, the data measured by the instrument can be used for analysis. In the present disclosure, profiling is performed by using a centWave algorithm in XCMS. The centWave algorithm is suitable for a high resolution mass spectrometer to collects data in a centroid mode. Another method in XCMS, Matched Filter algorithm, is suitable for a low resolution mass spectrometer to collect data in a centroid mode or in a profile mode, as shown in Table 6.

TABLE 6
XCMS algorithm centWave and MatchedFilter
applicable situations

| XCMS algorithm | Applicable for |
|---|---|
| centWave | High resolution mass spectrometer, for example, |
| | LC/{QTOF, OrbiTrap, FTICR}-MS to collect |
| | data in a centroid mode |
| MatchedFilter | Low resolution mass spectrometer to collect data |
| | in a profile mode |

The LC-MS/MS technology plays an important role in metabolomics, which can help us to identify metabolites in a non-targeted manner, thereby gaining a deeper understanding of metabolic processes in organisms. The LC-MS/MS technology achieves the detection and identification of metabolites by combining liquid chromatography (LC) and mass spectrometry (MS) techniques. This technology includes two main steps: liquid chromatography isolation and mass spectrometry analysis. In the liquid chromatography separation step, a mixed sample is separated by different tubular columns so as to separate different compounds. Next, these separated compounds are analyzed in a mass spectrometer. A mass spectrometry data collection method can adopt data dependency acquisition (DDA): this method can perform fragmentation analysis based on the top N strongest ions (m/z values) in MS1 scan, and then proceeds to the next N scans. This method can timely generate clean MS2 fragment map in real-time in the collection process, however, only a limited quantity of ions are fragmented, causing low coverage rate of detected metabolites and poor quantification accuracy of compounds. XCMS can process large-scale LC-MS/MS data to provide identification and quantitative information of metabolites. By XCMS, the present disclosure can detect metabolite characteristics from raw data for performing steps such as peak alignment, peak detection and qualitative analysis. Map comparison is performed by downloading MassBank of North America (MoNA) database and using MetaboAnnotation package to calculate similarity. The map with the similarity of 0.8 is considered as successful comparison. Subsequently, it can be cascaded from MoNA chemical annotation PubChem CID back to the work database and obtain chemical characteristic information.

Table 7 is a table showing the quantity of mans in each MS level

TABLE 7
Quantity table of Maps of each MS level in each group

| | STD_150 | T3 | T4 | T5 |
|---|---|---|---|---|
| MS1 | 13593 | 12257 | 11795 | 12483 |
| MS2 | 7051 | 12053 | 13864 | 11455 |

Table 8 shows parameters for centWave algorithm setting and corresponding results.

TABLE 8
Parameters and corresponding results
for centWave algorithm setting

| | STD_150 | T3 | T4 | T5 |
|---|---|---|---|---|
| Peakwidth | c (10.84, | c(16, | c(16.64, | c(14.4, |
| | 98.3) | 53) | 65) | 63.5) |
| Ppm | 11.792 | 16.55 | 15.05 | 9.5 |
| Noise | 0 | 0 | 0 | 0 |
| Snthresh | 10 | 10 | 10 | 10 |
| Mzdiff | 0.00428 | 0.00505 | 0.00153 | 0.00395 |
| Prefilter | c(3, 100) | c (3, 100) | c(3, 100) | c(3, 100) |
| Detecting | 11.79 ppm | 16.55 ppm | 15.05 ppm | 9.5 ppm |
| mass | | | | |
| traces at | | | | |
| Detecting | 60167 | 47788 | 55108 | 45112 |
| chroma- | | | | |
| tographic | | | | |
| peaks in # | | | | |
| of regions | | | | |
| # of found | 3183 | 10639 | 14375 | 9971 |

Chromatographic peak detection is performed aiming at MS2 level data. The results are as follows:
in STD_150 group:

##MSn data (Spectra) with 840 spectra in an MsBackendMzR backend:

| ## | msLevel | rtime | scanIndex |
|---|---|---|---|
| ## | <integer> | <numeric> | <integer> |
| ## | 2 | 56.7721 | 171 |
| ## | 2 | 69.7215 | 220 |
| ## | 2 | 56.6784 | 170 |
| ## | 2 | 57.9482 | 175 |
| ## | 2 | 70.2249 | 223 |
| . . . | . . . | . . . | . . . |
| ## | 2 | 5681.25 | 20520 |
| ## | 2 | 5691.79 | 20599 |
| ## | 2 | 5683.64 | 20538 |
| ## | 2 | 5694.22 | 20615 |
| ## | 2 | 5683.24 | 20533 |

In STD_150 group, a total of 840 MS2 maps are identified.
in T3 group:

##MSn data (Spectra) with 3160 spectra in an MsBackendMzR backend:

| ## | msLevel | rtime | scanIndex |
|---|---|---|---|
| ## | <integer> | <numeric> | <integer> |
| ## | 2 | 50.4434 | 162 |
| ## | 2 | 62.0200 | 259 |
| ## | 2 | 53.7112 | 188 |
| ## | 2 | 59.1386 | 232 |
| ## | 2 | 55.5188 | 201 |
| . . . | . . . | . . . | . . . |
| ## | 2 | 5591.22 | 23793 |
| ## | 2 | 5695.99 | 24290 |
| ## | 2 | 5593.66 | 23804 |
| ## | 2 | 5604.04 | 23857 |
| ## | 2 | 5691.46 | 24267 |

In T3 group, a total of 3160 MS2 maps are identified.
in T4 group:

##MSn data (Spectra) with 5267 spectra in a MsBackendMzR backend:

| ## | msLevel | rtime | scanIndex |
|---|---|---|---|
| ## | <integer> | <numeric> | <integer> |
| ## | 2 | 65.7560 | 307 |
| ## | 2 | 53.5262 | 200 |
| ## | 2 | 48.4590 | 162 |
| ## | 2 | 59.0258 | 244 |
| ## | 2 | 58.6508 | 240 |
| . . . | . . . | . . . | . . . |
| ## | 2 | 5693.87 | 25630 |
| ## | 2 | 5485.78 | 24661 |
| ## | 2 | 5496.56 | 24719 |
| ## | 2 | 5593.40 | 25201 |
| ## | 2 | 5603.85 | 25251 |

In T4 group, a total of 5267 MS2 maps are identified.
in T5 group:

##MSn data (Spectra) with 3058 spectra in a MsBackendMzR backend:

| ## | msLevel | rtime | scanIndex |
|---|---|---|---|
| ## | <integer> | <numeric> | <integer> |
| ## | 2 | 59.2191 | 232 |
| ## | 2 | 53.6207 | 197 |
| ## | 2 | 56.7521 | 218 |
| ## | 2 | 57.9279 | 225 |
| ## | 2 | 54.6040 | 206 |
| . . . | . . . | . . . | . . . |
| ## | 2 | 5636.09 | 23669 |
| ## | 2 | 5645.86 | 23712 |
| ## | 2 | 5656.09 | 23754 |
| ## | 2 | 5666.77 | 23798 |
| ## | 2 | 5693.03 | 23906 |

In T5 group, a total of 3058 MS2 maps are identified.

Table 9 shows results of mass spectrometry comparison of each group using MetaboAnnotation package

TABLE 9
Results of mass spectrometry comparison of
each group using MetaboAnnotation package

| | STD_150 | T3 | T4 | T5 |
|---|---|---|---|---|
| Total number | 71 | 491 | 352 | 371 |
| of matches | | | | |
| Number of | 840 (17 | 3160 (67 | 5267 (44 | 3058 (32 |
| query objects | matched) | matched) | matched) | matched) |
| Number of | 86576 (27 | 86576 | 86576 (118 | 86576 (83 |
| target objects | matched) | (99matched) | matched) | matched) |

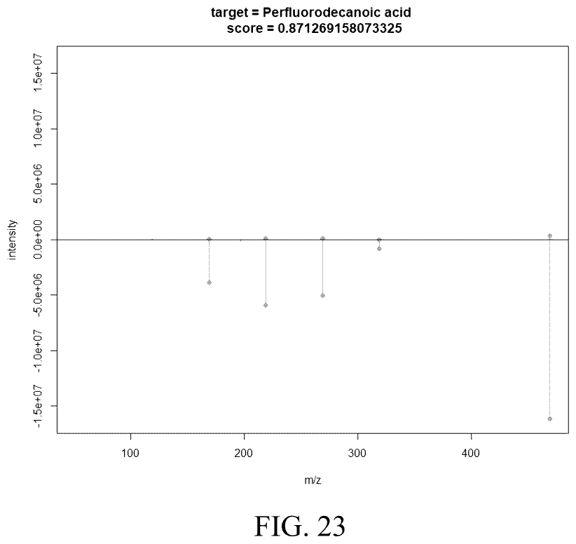

The present disclosure comprises preprocessing and parameter adjustment. Before wave peak identification, sample data is preprocessed, such as denoising, smoothing and background correction, for example, functions of noiseFilterGaussian( ), fillPeaks( ) and the like in XCMS are used. Multiple parameters need to be adjustment to use the XCMS software so as to achieve optimal effects. In the present disclosure, multiple instrument detection data are used to adjust parameters. Parameter adjustment has been completed for sample analysis, and a total ion chromatogram showing a charge-to-mass ratio of from 77.00029 to 1154.89783 is as shown in FIG. 23. FIG. 23 is a total ion chromatogram of a wastewater sample. In the present disclosure, optimized XCMS parameter adjustment is performed by IPO package.

IPO (Identification, Parameter optimization, and Optimization) is an R package for optimizing XCMS parameters, which can help researchers to better adjust the parameters of XCMS, thereby improving the analysis results of LC-MS/MS data. It takes effects in three aspects: identification: IPO helps recognition and identification of metabolites in liquid chromatography tandem mass spectrometry data by optimizing the parameters of XCMS. Through the adjustment of the parameters, IPO can better calibrate peak spectra and determine the starting and ending positions of peaks, thereby more accurately identifying metabolites. Parameter optimization: IPO automatically searches an optimal XCMS parameter combination by applying an optimization algorithm, thereby achieving the optimal data processing effect. This can save a lot of manual optimization time for relevant researchers, while ensuring the reliability and consistency of the analysis results. Optimization: IPO not only focuses on a single XCMS parameter, but also considers an impact relationship between multiple relevant parameters, thereby achieving overall analysis and optimization. Through the automatic search of the optimal parameter combination, IPO helps researchers to save time while improving the reliability of high-efficiency analysis and results. Through proper data preparation and IPO operation, metabolite information in liquid chromatography tandem mass spectrometry data can be more comprehensively understood.

Table 10 is a table showing optimal parameter setting in each group

TABLE 10
Table for optimal parameter setting in each group

| | STD_150 | T3 | T4 | T5 |
|---|---|---|---|---|
| min_peakwidth | 10.84 | 16 | 16.64 | 14.4 |
| max_peakwidth | 98.3 | 53 | 65 | 63.5 |
| ppm | 11.792 | 16.55 | 15.05 | 9.5 |
| mzdiff | 0.00428 | 0.00505 | 0.00153 | 0.00395 |
| snthresh | 10 | 10 | 10 | 10 |
| noise | 0 | 0 | 0 | 0 |
| prefilter | 3 | 3 | 3 | 3 |
| value_of_prefilter | 100 | 100 | 100 | 100 |

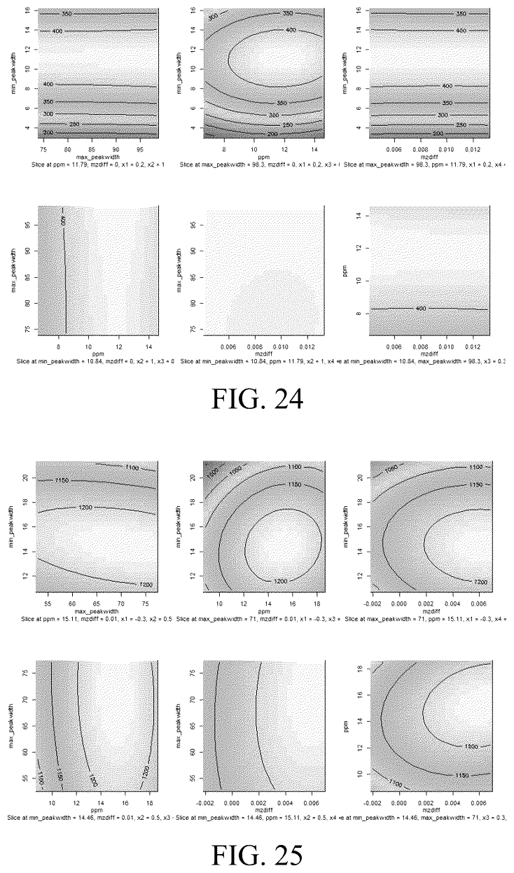

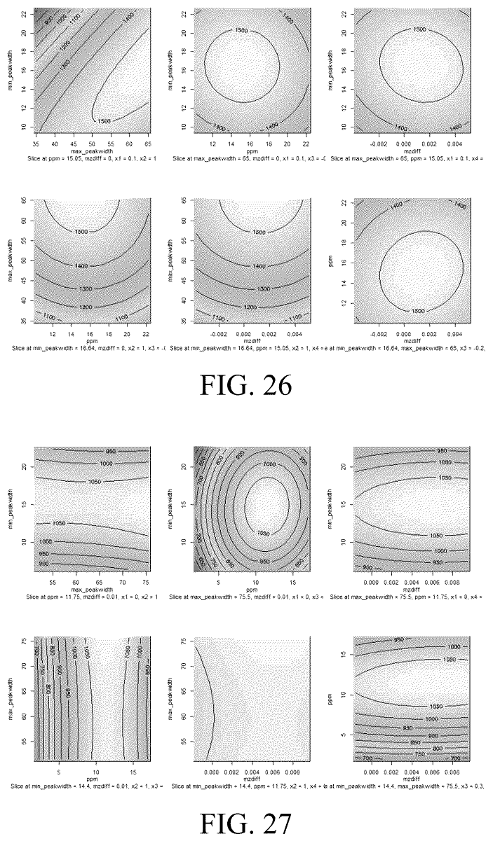

FIG. 24 shows optimized parameter test results under the condition of STD_150. FIG. 25 shows optimized parameter test results under the condition of T3. FIG. 26 shows optimized parameter test results under the condition of T4. FIG. 27 shows optimized parameter test results under the condition of T5.

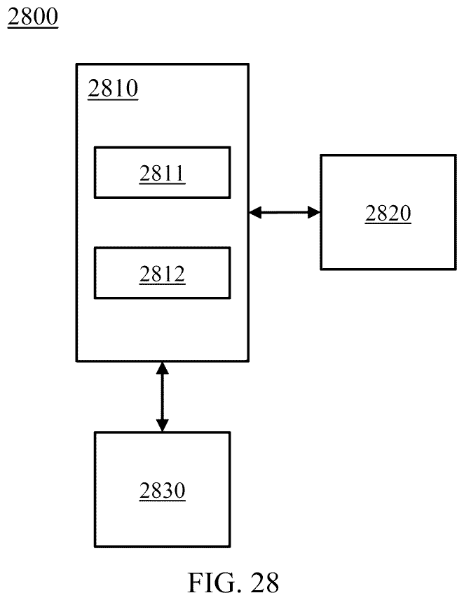

FIG. 28 shows a computer system 2800 for executing the method, software or operation system of the present disclosure. The computer system 2800 can comprise a host 2810, a database 2820 and an analyzer 2830. The host 2810 can comprise a processor 2811 and a memory 2812. The memory 2812 may store indications to allow the processor 2811 to execute the method, software or operation system of the present disclosure. The database 2820 can be communicated with the host 2810. The database 2820 can store the database or data of the present disclosure. The analyzer 2830 can be communicated with the host 2810. The analyzer 2830 can analyze samples and transmit relevant data to the host 2810. The data transmitted by the analyzer 2830 can comprise data that have been converted and optimized. The data transmitted by the analyzer 2830 can comprise data that have not been converted and optimized, and the host 2810 can further convert and optimize data from the analyzer 2830.

In the present disclosure, the high-attention or control lists of various countries and international organizations are integrated in combination with several per/polyfluoroalkyl polyolefins databases and lists, and chemical information is integrated through chemical structure comparison to write application development interfaces (API) for fast access to characteristic information of specific chemicals. In the present disclosure, a PubChem molecular fingerprint group is established from simplified molecular input line entry specification (SMILES) of chemical structures, the above information is stored in the work database, and also the chemical characteristic interface that is easy to read is designed. In the present disclosure, a training dataset with 3810 pieces of information is organized based on carcinogenicity hazard endpoints. In the present disclosure, the QSAR model is designed based on the principle provided by OECD, the training dataset processing, hazard endpoints, algorithm and definition applicability domain are planned, a toxicity prediction model prototype is then established after seeking expert opinions through expert meeting, and the predicted chemical characteristics are input into the work database. For the instrument measurement and identification process, the conversion of the instrument raw data into public format, the instrument signal analysis process and parameter adjustment are completed in the present disclosure, and chemicals are actually detected and identified by test information.

As used herein, for the sake of description, space related terms such as “bottom”, “under”, “lower part”, “above”, “upper part”, “lower part”, “left” and “right” can be used herein to describe the relationship between one component or feature shown in the figure and another component or feature. In addition to the orientation shown in the figure, spatial related terms are intended to cover different orientations of the device in use or operation. The device can be oriented in other ways (rotating 90 degrees or being in other orientations), and the spatial related descriptive terms used herein can also be used for corresponding explanations. It should be understood that when a component is “connected” or “coupled” to another component, this component can be directly connected or coupled to another component, or there is an intermediate component.

As used in the present disclosure, the terms “approximately”, “essentially”, “generally” and “about” are used to describe and explain minor changes. When used in combination with events or situations, the term can refer to situations where the events or situations occur precisely, as well as situations where the events or situations occur approximately. As used herein, for a given value or range, the term “about” usually means being within ±10%, ±5%, ±1%, or ±0.5% of the given value or range. The range can be indicated herein as from one endpoint to the other end point, or between two endpoints. Unless otherwise specified, all scopes disclosed in the present invention include endpoints. The term “generally coplanar” can refer to two surfaces located within a few micrometers (μm) along the same plane, such as those located within 10 μm, 5 μm, 1 μm, or 0.5 μm along the same plane. When referring to values or characteristics that are generally the same, this term can refer to values within ±10%, ±5%, ±1%, or ±0.5% of an average value.

The above descriptions briefly illustrate several embodiments and detailed features of the present disclosure. The embodiments described in the present disclosure can be easily used as a basis for designing or modifying other programs and structures for achieving the same or similar objectives and/or obtaining the same or similar advantages introduced in the embodiments of the present disclosure. This type of equivalent construction does not depart from the spirit and scope of the present invention, and can be subject to various changes, substitutions, and modifications without departing from the spirit and scope of the present disclosure.

What is claimed is:

1. A method for chemical toxicity prediction, comprising:
receiving test data from an analysis instrument;
selecting a candidate chemical according to the test data;
according to a molecular fingerprint of the candidate chemical, predicting the toxicity of the candidate chemical by using a quantitative structure-activity relationship (QSAR) model.

2. The method according to claim 1, wherein:
the QSAR model comprises a support vector machine (SVM), a C5.0 decision tree model and a random forest.

3. The method according to claim 1, wherein
the candidate chemical belongs to a predicable range of the QSAR model;
the molecular weight of the candidate chemical is in a molecular weight interval of a training dataset of the QSAR model;
the average similarity of the candidate chemical is greater than the similarity (Tanimoto Coefficient) of the 95th percentile of the training dataset.

4. The method according to claim 1, wherein generating a training dataset comprises:
generating a combined database based on two chemical databases;
excluding inorganic compounds and metal organics in the combined database;
calculating corresponding similarity (Tanimoto Coefficient) according to the molecular fingerprint of each chemical item in the combined database and calculating the average similarity of the combined database; and
removing data of a chemical item from the combined database when a difference between the similarity of the chemical item and the average similarity is greater than a threshold.

5. The method according to claim 4, wherein generating the training dataset further comprises: removing repeated chemical items based on the molecular fingerprint of each chemical item in the combined database.

6. A system for chemical toxicity prediction, comprising:
a database;
a host comprising a memory and a processor; and
an analysis instrument,
wherein, the memory stores instructs so that the host executes the following operations:
receiving test data of a sample from the analysis instrument;
selecting a candidate chemical from the database according to the test data;
according to a molecular fingerprint of the candidate chemical, predicting the toxicity of the candidate chemical by using a quantitative structure-activity relationship (QSAR) model.
